SEQUENCE LISTING (33 sequences)

SEQ ID NO: 1 - DNA, 39 bp, Artificial
  Variable Heavy Chain Forward primer
ggtcagtcctctagatcttccgccgtgacgttggacgag

SEQ ID NO: 2 - DNA, 44 bp, Artificial
  Variable heavy Chain Reverse Primer
ctggccggcctggccactagtggaggagacgatgacttcggtcc

SEQ ID NO: 3 - DNA, 38 bp, Artificial
  Variable Ligh Chain Forward primer
gtggcccaggcggccctgactcagccgtcctcggtgtc

SEQ ID NO: 4 - DNA, 34 bp, Artificial
  Variable Light Chain Reverse Primer
ggaagatctagaggactgacctaggacggtcagg

SEQ ID NO: 5 - DNA, 42 bp, Artificial
  Overlap Extension Forward Primer
gaggaggaggaggaggaggtggcccaggcggccctgactcag

SEQ ID NO: 6 - DNA, 47 bp, Artificial
  Overlap Extension Reverse Primer
gaggaggaggaggaggaggagctggccggcctggccactagtggagg

SEQ ID NO: 7 - protein, 7 aa, Artificial
  CDRL2 region
XNTNRPS

SEQ ID NO: 8 - protein, 7 aa, Artificial
  CDRL2 region
DNTNRPS

SEQ ID NO: 9 - protein, 12 aa, Artificial
  CDRL3 region
GXXDXSXXXXXX

SEQ ID NO: 10 - protein, 12 aa, Artificial
  CDRL3 region
GXXDXSAXXXXI

SEQ ID NO: 11 - protein, 12 aa, Artificial
  CDRL3 region
GSYDRSAGYVGI

SEQ ID NO: 12 - protein, 9 aa, Artificial
  CDRL1 region
XXXXXXXYG

SEQ ID NO: 13 - protein, 9 aa, Artificial
  CDRL1 region
SGGXXSXYG

SEQ ID NO: 14 - protein, 9 aa, Artificial
  CDRL1 region
SGGGGSYYG

SEQ ID NO: 15 - protein, 10 aa, Artificial
  CDRH1 region
GFXFXXXXMX

SEQ ID NO: 16 - protein, 10 aa, Artificial
  CDRH1 region
GFTFXSXXMX

SEQ ID NO: 17 - protein, 10 aa, Artificial
  CDRH1 region
GFTFDSYAMY

SEQ ID NO: 18 - protein, 14 aa, Artificial
  CDRH2 region
IXXXGXXTXXGAAV

SEQ ID NO: 19 - protein, 14 aa, Artificial
  CDRH2 region
INRFGXSTGHGAAV

SEQ ID NO: 20 - protein, 19 aa, Artificial
  CDRH3 region
SXXGXXXXXXXXXXXXIDA

SEQ ID NO: 21 - protein, 19 aa, Artificial
  CDRH3 region
SVHGXCASGXWCSXASIDA

SEQ ID NO: 22 - protein, 94 aa, Artificial
  Variable Light Chain - Clone AE8
GGTVKITCSGGGGSYYGWFQQKSPGSAPVTVIYDNTNRPSNIPSRFSGSLSGSTNTLTITGVQAEDEAVYYCGSYDRSAGYVGIFGAGTTLTVL

SEQ ID NO: 23 - protein, 91 aa, Artificial
  Variable Heavy Chain - AE8
AVTLDESGGGLQTPGGGLSLVCKASGFTFDSYAMYWVRQAPGKGLEWVASINRFGSSTGHGAAVKGRATISRDNGQSTLGAYPYDVPDYAS

SEQ ID NO: 24 - protein, 89 aa, Artificial
  Variable Light Chain - Clone AE9
GGTVKITCSGGGGSYYGWFQQKSPGSAPVTVIYDNTNRPSNIPSRFSGSKSGSTGTLTITVQAEDEAVYYCGNFDTSAIFGAGTTLTVL

SEQ ID NO: 25 - protein, 153 aa, Artificial
  Variable Heavy Chain - AE9
AVTLDESGGGLQTPGGALSLICKASGFTFSSFNMIWVRQAPGKGLEFVGSINRFGNSTGHGAAVKGRVTISRDDGQSTVRLQLNNLRAEDTGTYFCAKSVHGHCASGYWCSAASIDAWGHGTEVIVSSTSGQAGQHHHHHHGAYPYDVPDYAS

SEQ ID NO: 26 - protein, 90 aa, Artificial
  Variable Light Chain - CD3
GGTVEITCSGGSYSYGWYQQKSPGSAPVTVIYQNTNRPSDIPSRFSGSKSGSTGTLTITGVRAEDEAVYYCGSFDSSVGMFGAGTTLTVL

SEQ ID NO: 27 - protein, 148 aa, Artificial
  Variable Heavy Chain - Clone CD3
AVTLDESEGGLQTPGGALSLVCKASGFSFSDRGMHWVRQAPGKGLEYVAGIYDDGGTTYYGAAVKGRASITRDNGQSAVRLQLNNLRAEDTATYYCAKSAAGDAWGADDIDAWGHGTEVIVSSTSGQAGQHHHHHHGAYPYDVPDYAS

SEQ ID NO: 28 - protein, 79 aa, Artificial
  Variable Light Chain - Clone CD11
KWYGWYQQKAPGSAPVTLIYDNTNRPSDIPSRFSGSASGSTATLTITGVQVEDEAVYFGGYDGSTDAGIFGAGTTLTVL

SEQ ID NO: 29 - protein, 153 aa, Artificial
  Variable Heavy Chain - Clone CC11
AVTLDESGGGLQTPGGALSLVCKASGFDFSSYQMNWIRQAPGKGLEWVAAINKFGTSTSRGAAVKGRVTISRDDGQSTVRLQLNNLRSEDTATYFCAKSAYGSCASGSWCSAASIDAWGHGTEVIVSSTSGQAGQHHHHHHGAYPYDVPDYAS

SEQ ID NO: 30 - protein, 192 aa, Artificial
  Clone  AE8 scFv
GGTVKITCSGGGGSYYGWFQQKSPGSAPVTVIYDNTNRPSNIPSRFSGSLSGSTNTLTITGVQAEDEAVYYCGSYDRSAGYVGIFGAGTTLTVLGQSSRSSAVTLDESGGGLQTPGGGLSLVCKASGFTFDSYAMYWVRQAPGKGLEWVASINRFGSSTGHGAAVKGRATISRDNGQSTLGAYPYDVPDYAS

SEQ ID NO: 31 - protein, 249 aa, Artificial
  Clone AE9 scFv
GGTVKITCSGGGGSYYGWFQQKSPGSAPVTVIYDNTNRPSNIPSRFSGSKSGSTGTLTITVQAEDEAVYYCGNFDTSAIFGAGTTLTVLGQSSRSSAVTLDESGGGLQTPGGALSLICKASGFTFSSFNMIWVRQAPGKGLEFVGSINRFGNSTGHGAAVKGRVTISRDDGQSTVRLQLNNLRAEDTGTYFCAKSVHGHCASGYWCSAASIDAWGHGTEVIVSSTSGQAGQHHHHHHGAYPYDVPDYAS

SEQ ID NO: 32 - protein, 245 aa, Artificial
  Clone CD3 scFv
GGTVEITCSGGSYSYGWYQQKSPGSAPVTVIYQNTNRPSDIPSRFSGSKSGSTGTLTITGVRAEDEAVYYCGSFDSSVGMFGAGTTLTVLGQSSRSSAVTLDESEGGLQTPGGALSLVCKASGFSFSDRGMHWVRQAPGKGLEYVAGIYDDGGTTYYGAAVKGRASITRDNGQSAVRLQLNNLRAEDTATYYCAKSAAGDAWGADDIDAWGHGTEVIVSSTSGQAGQHHHHHHGAYPYDVPDYAS

SEQ ID NO: 33 - protein, 240 aa, Artificial
  Clone CD11 scFv
KWYGWYQQKAPGSAPVTLIYDNTNRPSDIPSRFSGSASGSTATLTITGVQVEDEAVYFCGGYDGSTDAGIFGAGTTLTVLGQSSRSSAVTLDESGGGLQTPGGALSLVCKASGFDFSSYQMNWIRQAPGKGLEWVAAINKFGTSTSRGAAVKGRVTISRDDGQSTVRLQLNNLRSEDTATYFCAKSAYGSCASGSWCSAASIDAWGHGTEVIVSSTSGQAGQHHHHHHGAYPYDVPDYAS

(19) United States
(12) Patent Application Publication
(10) Pub. No.: US 2011/0034676 A1
(43) Pub. Date: Feb. 10, 2011

(54) ANTI-SIALIC ACID ANTIBODY MOLECULES

(75) Inventors: Gerard Gary Donohoe, Kimmage, Dublin (IE); Barry Byrne, Artane, Dublin (IE); Stephen Henry Hearty, Dundalk (IE); Richard O'Kennedy, Dublin (IE)

Correspondence Address: KNOBBE MARTENS OLSON & BEAR LLP, 2040 MAIN STREET, FOURTEENTH FLOOR, IRVINE, CA, 92614, US

(73) Assignee: DUBLIN CITY UNIVERSITY, Glasnevin, Dublin (IE)

(21) Appl. No.: 12/852,354

(22) Filed: Aug. 6, 2010

(30) Foreign Application Priority Data
Aug. 7, 2009 (EP) 09167495.2

(51) Int. Cl. C07K 16/00; C07K 14/00

(52) U.S. Cl. 530/387.3; 530/350

(57) ABSTRACT

A method of generating and isolating a recombinant high affinity anti-sialic acid antibody molecule comprises the steps of immunising a host with an immunogen comprising a conjugate of sialic acid and a carrier protein to generate an anti-sialic acid polyclonal serum, isolating a sample of RNA from the immunised avian host, and generating and screening of a library of recombinant antibody molecules from the RNA sample, and isolating a recombinant high affinity anti-sialic acid antibody molecule. The antibody molecule is selected from the group consisting of: whole antibodies; scFv fragments; and Fab fragments, and the host is Gallus domesticus. A recombinant avian antibody fragment having high binding affinity to sialic acid and obtainable by the method of the invention, and an anti-sialic acid polyclonal serum obtainable by immunising an avian host with a conjugate of sialic acid and carrier protein, are also described.

### CROSS-REFERENCE TO RELATED APPLICATIONS

This application claims the benefit of European Patent Application Number 09167495.2 filed on Aug. 7, 2009, the disclosure of which is hereby expressly incorporated by reference in its entirety and hereby expressly made a portion of this application.

### BACKGROUND OF THE INVENTION

1. Field of the Invention

The invention relates to a method of generating a library of recombinant anti-sialic acid antibody molecules, and anti-sialic acid antibody molecules obtainable by the method of the invention. The invention also relates to a conjugate useful in immunising a host to generate a polyclonal anti-sialic acid serum.

2. Description of the Related Art

Sialic acids are acidic monosaccharides that reside as terminal monosaccharides on N- and O-linked glycans. They are actively involved in a plethora of biological phenomena, ranging from cell-cell adhesion and recognition, intracellular signaling events, pathogen attack, viral infection, inflammatory disease and cancer. These moieties belong to a family of α-keto acids that are structurally characterised by the presence of a nine-carbon backbone. The three major forms of sialic acid are Neu5Ac, Neu5Gc and 2-keto-3-deoxy-D-glycero-D-galacto-nononic acid (KDN). Neu5Ac is the most ubiquitous sialic acid, in eukaryotic cells it is α(2,3) or α(2,6) linked to galactose and α(2,6)-linked to N-acetyl-galactosamine (GalNAc). Sialic acid moieties may also interact with each other, predominantly through α(2,8) and, to a lesser extent, α(2,9) linkages to form polysialic acid (PSA, or colominic acid). In addition to these basic forms, more than 50 distinct sialic acid structures have been identified in nature, arising from acetylation, methylation, lactylation, sulfation, and phosphorylation of the C-4, C-5, C-7, C-8, or C-9 hydroxyl groups.^{1,2,3,4,5 and 6} A number of sensitive methods have been published that either measure total sialic acid content or different sialic acid types. However, these methods are time consuming, costly, laborious and may require sophisticated instrumentation as well as a high degree of operator knowledge for implementation. Hence, these assay formats are not ideally suited for routine use with complex sample types. The colorimetric determination of sialic acid, for example uses orcinol, resorcinol, periodic and thiobarbituric acid. These reagents are quite toxic and a prerequisite for performing this assay is the availability of a purified sample as lipid or 2-deoxyribose contamination generates erroneous results.

Lectin-based assays are available for certain types of sialic acid (Sambucus nigra and Maackia amurensis-derived lectins for the detection of α(2,3) and α(2,6)-linked sialic acid, respectively). However, the use of some lectins for assay development is hindered by their low sensitivity and poor specificity. The detection of sialic acid in the context of a sialoglycoprotein or as a free moiety may be facilitated by a panel of sensitive analytical techniques. These include high-performance liquid chromatography (HPLC), gas-chromatography combined with mass spectrometry (GC-MS), nuclear magnetic resonance spectrometry (NMR) and capillary electrophoresis (CE). However, these methods require significant sample preparation, specialised equipment, purification of the target protein and often require lengthy and complex data analysis for monitoring sialylation.

### SUMMARY

The methods of the invention are directed to the generation of recombinant, anti-sialic acid, antibody molecules that have high affinity binding to sialic acid. The method involves immunising a host with a synthetic conjugate, namely a conjugate of sialic acid and a carrier protein, in which the conjugate comprises a plurality of sialic acid molecules conjugated to one carrier protein molecule. The host and conjugate are specifically chosen such that the host glycome is deficient in the sialic acid present in the conjugate. The method of the invention generally involves immunising a host with the conjugate to raise a polyclonal serum, obtaining a sample of RNA from the thus-immunised host, generating a library of recombinant antibody molecules, and then screening the library to isolate a clone having high affinity binding to sialic acid. The methods of the invention result in the generation of anti-sialic acid antibody molecules having nanamolar binding affinity to sialic acid. Moreover, the use of a synthetic conjugate of a sialic acid and a carrier protein (i.e. a sialylated neoglycoprotein) induces the non-natural pairing of variable heavy and variable light antibody chains in immunised hosts, which would not be possible using a natural immunogen.

According to the invention, there is provided a method of generating a library of recombinant high affinity anti-sialic acid antibody molecules, the method comprising the steps of:
immunising a host with an immunogen comprising a conjugate of sialic acid and a carrier protein to generate an anti-sialic acid polyclonal serum, wherein the host glycome is deficient in the sialic acid;
isolating a sample of RNA from the immunised avian host; and
generating and screening a cDNA library of recombinant antibody molecules constructed from the RNA sample.

In another aspect, the invention relates to a method of generating a recombinant antibody molecule having high-affinity binding to sialic acid, the method including the steps of:
immunising a host with an immunogen comprising a conjugate of sialic acid and a carrier protein to generate an anti-sialic acid polyclonal serum, wherein the host glycome is deficient in the sialic acid;
isolating a sample of RNA from the immunised host;
generating and screening a cDNA library of recombinant antibody molecules constructed from the RNA sample; and
isolating a clone having high-affinity binding to sialic acid.

The methods of the invention involve immunising a host which is deficient in the sialic acid that is part of the immunogen. Thus, when the sialic acid is Neu5Gc, the host is an organism that is naturally deficient in Neu5Gc, i.e. a host that does not contain Neu5Gc as part of its natural glycome, or a host that is genetically engineered to be deficient in Neu5Gc. An example of host that is naturally deficient in Neu5Gc is an avian host, especially an avian host of the Gallus family, for example Gallus domesticus.

Various techniques are available for the generation and screening of a library of recombinant antibody molecules from the RNA sample, including phage display, ribosomal display, yeast display and bacterial display. A number of these techniques are described in Adv Drug Deliv Rev. 2006 Dec. 30; 58(15): 1622-1654 and Nature Biotechnology Volume 23, No: 9 (9 Sep. 2005). In a preferred embodiment, phage display is employed for the generation and screening of a library of recombinant antibody molecules, although the methods of the invention are not restricted to the use of phage display.

The carrier protein is typically a protein which is capable of being conjugated to a plurality of sialic acid molecules, suitably demonstrates low immunogenicity, and ideally is well characterised. In one embodiment, the carrier protein comprises any serum albumin protein, generally bovine serum albumin (BSA) or human serum albumin (HSA), conjugated to at least one sialic acid molecule. Suitably, then sialic acid is Neu5Gc or Neu5Ac, ideally Neu5Gc. Suitably, the sialic acid is conjugated to the carrier protein by a linker. Preferably, the linker is a hydrocarbon chain having at least five, six, seven, eight, nine or ten carbon atoms. Ideally, a plurality of sialic acid molecules is conjugated to a single carrier protein, typically via linker molecules. In a particularly preferred embodiment, at least 2, 3, 4, 5, 6, 7, 8, 9, 10, 12, 14, 15, 17, 19 or 20 sialic acid molecules are conjugated to a single carrier protein molecule. In one embodiment, the conjugate has a formula shown in FIG. 1C, in which XSA is a serum albumin protein, suitably selected from the group consisting of: HSA; and BSA.

In this specification, the term “sialic acid” should be understood as meaning Neu5Gc or Neu5Ac, or both. Thus, the anti-sialic acid antibody fragments generated according to the methods of the invention will have specific binding affinity for Neu5Gc, Neu5Ac, or ideally both Neu5Gc and Neu5Ac.

The terms “high binding affinity to sialic acid” should be understood to mean a binding affinity to sialic acid (ideally both Neu5Gc and Neu5Ac) of at least micromolar affinity, ideally at least nanomolar affinity, as determined by Biacore kinetic analysis. Likewise, the term “high affinity” should be understood to mean a binding affinity to sialic acid (ideally both Neu5Gc and Neu5Ac) of at least micromolar affinity, ideally at least nanomolar affinity, as determined by Biacore kinetic analysis.

The antibody molecules generated according to the methods of the invention are selected from the group consisting of: whole antibodies, and antibody fragments, for example scFv fragments or Fab fragments. In one embodiment of the invention, the RNA sample derived from the host is obtained from a biological sample selected from the group consisting of: bone marrow; and spleen. Ideally, the RNA sample is obtained from both the bone marrow and spleen cells.

In an embodiment in which phage display is employed, the methods of the invention provide a library of clones, each clone comprising a phage displaying a recombinant anti-sialic acid antibody molecule. Clones expressing high affinity antibody molecules (for example, antibody fragments that bind sialic acid, especially Neu5Gc, Neu5Ac, or ideally both) are generally isolated by means of biopanning against decreasing concentrations of sialic acid. Ideally, sialic acid is immobilised during the biopanning process. Antibody fragments are produced by means of amplification in a suitable producer cell, for example, Escherichia coli.

In a preferred embodiment of the invention, the host is an avian host, typically of the Gallus family. Ideally, the avian host is Gallus domesticus. However, other suitable avian hosts will be apparent to the skilled person, especially other related species that do not contain Neu5Gc as part of its glycome (either naturally deficient, or genetically engineered to be deficient)

The library construction process involves the use of variable heavy chain (V_{H}) primers, variable light chain (V_{L}) primers, and overlap extension primers. Suitably, the V_{H} primers are the primers shown in SEQ ID NO'S 1 and 2. Suitably, the V_{L}, primers are the primers shown in SEQ ID NO'S 3 and 4. Suitably, the overlap extension primers are the primers shown in SEQ ID NO'S 5 and 6.

The invention also relates to a phage display library obtainable by the method of the invention (in which all or most of the phage, display a high affinity recombinant anti-sialic acid antibody molecule).

The invention also relates to a library of recombinant anti-sialic acid antibody molecules obtainable by means of the method of the invention.

The invention also relates to a recombinant antibody fragment having high binding affinity to sialic acid, ideally to both Neu5Gc and Neu5Ac.

The invention also relates to an anti-sialic acid polyclonal serum obtainable by immunising a host with a conjugate of sialic acid and carrier protein, ideally a conjugate of Neu5Gc or NeuAc and a carrier protein, in which the host glycome is deficient in the sialic acid. Typically, the carrier protein is BSA or HSA. Ideally, the conjugate comprises at least 2, 4, 6, 8, 10, 12, 15, 18 or 20 sialic acid molecules conjugated to one molecule of carrier protein.

The invention also relates to a recombinant anti-sialic acid antibody molecule, typically a recombinant avian anti-sialic acid antibody molecule.

In this specification, the term “anti-sialic acid” should be understood as meaning to have a binding affinity to Neu5Gc or Neu5Ac (or both) of at least micromolar, and ideally at least nanomolar, affinity, as determined by Biacore kinetics. Ideally, the antibody is both anti-Neu5Gc and anti-Neu5Ac.

Ideally, the antibody molecule is an antibody fragment, typically a Fab or scFv fragment.

In one embodiment, the antibody has a CDRL2 region of amino acid sequence XNTNRPS (SEQ ID NO: 7), ideally DNTNRPS (SEQ ID NO: 8). These sequences are consensus sequences from the CDRL2 regions of clones AE8, AG9, CD3, and CC11.

In one embodiment, the antibody has a CDRL3 region of amino acid sequence GXY/FDXSXXXXXX (SEQ ID NO: 9), preferably GXXDXSAXXXXI (SEQ ID NO: 10), and ideally GSYDRSAGYVGI (SEQ ID NO: 11). These sequences are consensus sequences from the CDRL3 regions of clones AE8, AG9, CD3, and CC11.

In one embodiment, the antibody has a CDRL1 region of amino acid sequence XXXXXXXYG (SEQ ID NO: 12), suitably SGGXXSXYG (SEQ ID NO: 13), and ideally SGGGGSYYG (SEQ ID NO: 14). These sequences are consensus sequences from the CDRL1 regions of clones AE8, AG9, CD3, and CC11.

In one embodiment, the antibody has a CDRH1 region of amino acid sequence GFXFXXXXMX (SEQ ID NO: 15), suitably GFTFXSXXMX (SEQ ID NO: 16), and ideally GFTFDSYAMY (SEQ ID NO: 17). These sequences are consensus sequences from the CDRH1 regions of clones AE8, AG9, CD3, and CC11.

In one embodiment, the antibody has a CDRH2 region of amino acid sequence IXXXGXXTXXGAAV (SEQ ID NO: 18), suitably INRFGS/NSTGHGAAV (SEQ ID NO: 19). These sequences are consensus sequences from the CDRH2 regions of clones AE8, AG9, CD3, and CC11.

In one embodiment, the antibody has a CDRH3 region of amino acid sequence SXXGXXXXXXXXXXXXIDA (SEQ ID NO: 20), suitably SVHGS/HCASGT/YWCSP/AASIDA (SEQ ID NO: 21). These sequences are consensus sequences from the CDRH3 regions of clones AE8, AG9, CD3, and CC11.

In the sequence listings above, “X” denotes either any amino acid, or no amino acid. Further, “X/D” denotes that the residue at that position is X (as defined above) or D.

In one preferred embodiment of the invention, the antibody comprises a polypeptide having a CDRL1 region according to SEQ ID NO: 12, 13 or 14, a CDRL2 region according to SEQ ID NO: 7 or 8, and a CDRL3 region according to SEQ ID NO'S: 9, 10 or 11, or a functional variant of the polypeptide.

In one preferred embodiment of the invention, the antibody comprises a polypeptide having a CDRH1 region according to SEQ ID NO: 15, 16 OR 17, a CDRH2 region according to SEQ ID NO: 18 or 19, and a CDRH3 region according to SEQ ID NO'S: 20 or 21, or a functional variant of the polypeptide.

In a particularly preferred embodiment of the invention, the antibody comprises a polypeptide having a CDRL1 region according to SEQ ID NO: 12, 13 or 14, a CDRL2 region according to SEQ ID NO: 7 or 8, a CDRL3 region according to SEQ ID NO'S: 9, 10 or 11, a CDRH1 region according to SEQ ID NO: 15, 16 or 17, a CDRH2 region according to SEQ ID NO: 18 or 19, and a CDRH3 region according to SEQ ID NO'S: 20 or 21, or a functional variant of the polypeptide. Ideally, the antibody is an antibody fragment, typically an scFv antibody.

The invention also provides an antibody molecule comprising a heavy chain variable region and a light chain variable region, the light chain variable region comprising a CDRL1 region according to SEQ ID NO: 12, 13 or 14, a CDRL2 region according to SEQ ID NO: 7 or 8, a CDRL3 region according to SEQ ID NO'S: 9, 10 or 11, the heavy chain variable region comprising a CDRH1 region according to SEQ ID NO: 15, 16 or 17, a CDRH2 region according to SEQ ID NO: 18 or 19, and a CDRH3 region according to SEQ ID NO'S: 20 or 21.

The invention also provides an antibody molecule comprising a heavy chain variable region and a light chain variable region, the light chain variable region comprising a CDRL1 region according to SEQ ID NO: 14, a CDRL2 region according to SEQ ID NO: 8, a CDRL3 region according to SEQ ID NO: 11, the heavy chain variable region comprising a CDRH1 region according to SEQ ID NO: 17, a CDRH2 region according to SEQ ID NO: 19, and a CDRH3 region according to SEQ ID NO: 21.

The invention also provides an antibody molecule comprising a heavy chain variable region and a light chain variable region, the light chain variable region comprising a sequence according to SEQ ID NO: 22, or a functional variant thereof, and the heavy chain variable region comprising a sequence according to SEQ ID NO: 23, or a functional variant thereof.

The invention also provides an antibody molecule comprising a heavy chain variable region and a light chain variable region, the light chain variable region comprising a sequence according to SEQ ID NO: 24, or a functional variant thereof, and the heavy chain variable region comprising a sequence according to SEQ ID NO: 25, or a functional variant thereof.

The invention also provides an antibody molecule comprising a heavy chain variable region and a light chain variable region, the light chain variable region comprising a sequence according to SEQ ID NO: 26, or a functional variant thereof, and the heavy chain variable region comprising a sequence according to SEQ ID NO: 27, or a functional variant thereof.

The invention also provides an antibody molecule comprising a heavy chain variable region and a light chain variable region, the light chain variable region comprising a sequence according to SEQ ID NO: 28, or a functional variant thereof, and the heavy chain variable region comprising a sequence according to SEQ ID NO: 29, or a functional variant thereof.

The invention also relates to an antibody molecule comprising a sequence selected from the group consisting of: SEQ ID NO: 30; 31; 32; and 33.

Suitably, the antibody molecule is an scFv antibody fragment in which the light chain variable region and heavy chain variable region are connected in series in a single molecule, usually by means of a linker. However, the antibody molecule may also be in the form of a Fab antibody fragment.

In this specification, the term “functional variant” should be understood as meaning a variant of the antibody-related polypeptide having at least 80%, 90%, and ideally at least 95%, 96%, 97%, 98% or 99% SEQ IDentity (homology) with the listed polypeptides and which have a binding affinity for sialic acid that is not less than 80% that of the listed polypeptides, and ideally not less than 90% that of the listed polypeptides. The term should be taken to include antibody molecules that are altered in respect of one or more amino acid residues. Preferably such alterations involve the insertion, addition, deletion and/or substitution of 5 or fewer amino acids, more preferably of 4 or fewer, even more preferably of 3 or fewer, most preferably of 1 or 2 amino acids only. Insertion, addition and substitution with natural and modified amino acids is envisaged. The variant may have conservative amino acid changes, wherein the amino acid being introduced is similar structurally, chemically, or functionally to that being substituted. In this context, sequence homology comprises both SEQ IDentity and similarity, i.e. an antibody molecule that shares 70% amino acid homology with a listed is one in which any 70% of aligned residues are either identical to, or conservative substitutions of, the corresponding residues in the listed antibody molecule.

The term “variant” is also intended to include chemical derivatives of the listed antibody molecules, i.e. where one or more residues of the antibody molecule is chemically derivatised by reaction of a functional side group. Also included within the term variant are antibody molecules in which naturally occurring amino acid residues are replaced with amino acid analogues.

Antibody molecules (including variants and fragments thereof) of and for use in the invention may be generated wholly or partly by phage display, chemical synthesis or by expression from nucleic acid. The proteins and peptides of and for use in the present invention can be readily prepared according to well-established, standard liquid or, preferably, solid-phase peptide synthesis methods known in the art (see, for example, J. M. Stewart and J. D. Young, Solid Phase Peptide Synthesis, 2nd edition, Pierce Chemical Company, Rockford, Ill. (1984), in M. Bodanzsky and A. Bodanzsky, The Practice of Peptide Synthesis, Springer Verlag, New York (1984).

The invention also relates to a conjugate of sialic acid to a carrier protein. Typically, the sialic acid is Neu5Gc or Neu5Ac. Suitably, the carrier protein is a protein that is capable of being conjugated to a plurality of sialic acid molecules, for example at least 2, 3, 4, 5, 6, 7, 8, 9, 10, 11, 12, 13, 14, 15, 16, 17, 18, 19 or 20 sialic acid molecules. Preferably, the carrier protein is one which when conjugated to a sialic acid mimics a sialylated neoglycoprotein. In one embodiment, the carrier protein is a serum albumin, typically HSA or BSA. Typically, the carrier protein is conjugated to a plurality of sialic acid molecules, for example at least 2, 3, 4, 5, 6, 7, 8, 9, 10, 11, 12, 13, 14, 15, 16, 17, 18, 19 or 20 sialic acid molecules. Ideally, the or each sialic acid molecule is conjugated to the carrier protein by means of a linker. Suitably, the linker is a hydrocarbon chain having at least five, six, seven, eight, nine or ten carbon atoms in the backbone, and optionally comprises a peptide bond intermediate the ends of the chain. Ideally, the conjugate has the formula shown in FIG. 1C, in which XSA is a serum albumin protein, suitably selected from the group consisting of: HSA; and BSA.

The invention also relates to the use of a conjugate of the invention as an immunogen in the generation of anti-sialic acid antibodies.

The invention also relates to a method of identifying and/or quantifying the presence of a sialic acid in a sample comprising the steps of reacting an antibody molecule of the invention with the sample, and then detecting an immuno-specific reaction between the antibody molecule and sialic acid.

The invention also relates to a method of isolating sialic acid from a sample comprising the steps of reacting an antibody molecule of the invention with the sample to form an antibody-sialic acid complex, and then separating the complex from the sample. Typically, the antibody molecule of the invention is immobilised to a support, for example a well of a microtitre plate, or a stationary phase of a chromatography column.

The invention also relates to an ELISA kit suitable for identifying and/or quantifying the presence of a sialic acid in a sample, the kit comprising an antibody molecule of the invention immobilised to a support, and optionally one or more diagnostic reagents capable of detecting and/or quantifying any immunospecific reaction between the antibody molecule and sialic acid.

The invention also relates to the use of an antibody molecule of the invention as a targeting vector for targeting a molecule to a specific locus in a sample. The molecule to be targeted may be, for example, a pharmaceutically active agent such as an anti-cancer drug or a drug suitable for treatment of a neurological disorder, or an imaging molecule such as a dye. The sample may be a cell, tissue, an organ or a body. Thus, the use is intended for both in-vitro, in-vivo, and ex-vivo applications, and for the purpose of therapy, diagnosis, and research.

### BRIEF DESCRIPTION OF THE DRAWINGS

FIG. 1: The synthesis of the BSA and HSA-Neu5Gc containing conjugates. A batch of a donor derivative (compound A) of Neu5Gc was synthesised from the free sugar. Compound A was further derivatised to form a new derivative with an activated ester at the end of a long chain (compound B). Compound B was reacted with the side chain amines of the protein's (HSA/BSA) lysine residues, forming amide bonds with the sugar derivative (compound C). The pre-activated form of compound B was checked by NMR to ensure structural correctness and by TLC to determine purity level. The final product, compound C, was checked by MALDI-MS to give an accurate figure for the degree of sugar substitution to the protein.

FIG. 2a: Titre of an avian antiserum response to a Neu5Gc-BSA conjugate.

FIG. 2b: Titre of an avian antiserum response to a Neu5Gc-polyacrylamide conjugate.

FIG. 2c: Inhibition ELISA of the avian antiserum with free Neu5Gc-BSA conjugate. The serum-based antibodies were able to compete between free and immobilised Neu5Gc-BSA, verifying that the response was sialic acid-specific.

FIG. 3a: Soluble monoclonal phage ELISA of competitively-eluted phage clones from rounds three and four of biopanning. The ELISA threshold is marked with a horizontal line. The BSA bar represents the total average binding of all clones to BSA. A selection of positive clones is illustrated.

FIG. 3b: Soluble monoclonal phage ELISA of trypsin-eluted phage clones from rounds three and four of biopanning. The ELISA threshold is marked with a horizontal line. The BSA bar represents the total average binding of all clones to BSA.

FIG. 3c: Soluble anti-sialic acid clones that recognise Neu5Gc in the form of both Neu5Gc-BSA and Neu5Gc-PAA. The ELISA threshold is marked with a horizontal line. The BSA bar represents the total average binding of all clones to BSA. A small selection of positive clones is shown.

FIG. 4: A cross reaction study of soluble anti-Neu5Gc scFvs to different monosaccharides and sialic acids. Clone AE8 can recognise sialic acid in the context of Neu5Ac, Neu5Gc and polysialic acid (PSA). Moreover, no significant binding to galactose or glucose was observed.

FIG. 5: Comparative sequence analysis of five different sialic acid-binding clones. All clones were sequenced in triplicate. All five scFv genes are different, clone CD3 shows the greatest differences in both the heavy and light chain regions. AE8 and AG9 show only subtle differences in CDRL3, CDRH1, CDRH2 and CDRH3 regions.

FIG. 6: SEC-HPLC analysis of IMAC-purified AE8. The protein standards are represented by the black solid line. A neat sample of purified AE8 (represented by the black dotted line) in 1×PBS (pH 7.2) was applied to the HPLC column. A Phenomenex 3000 SEC column was used with 1×PBS (pH 7.2) mobile phase at a flow rate of 0.5 ml/minute and monitored by UV absorbance at 280 nm. The scFv dimer peak was observed at 16.6 minutes and a smaller monomer peak eluted at 17.7 minutes.

FIG. 7: FPLC analysis of the IMAC-purified anti-sialic acid scFv (AE8). 100 μl of the AE8 sample was applied to a HiLoad™ 16/60 Superdex™ 200 Prep-grade FPLC column using filtered and degassed 1×PBS (pH 7.4) at a flow rate of 1 ml/min. The retention time of the calibration standards indicated are Bovine Thyroglobulin, 670 kDa, 51.09 min; Human gamma globulin, 150 kDa, 67.15 min; Ovalbumin, 44 kDa, 82.03 min and Myoglobin, 17 kDa, 93.65 min. The coefficient of determination for the standard curve (y=−0.034x+7.716) was R²=0.9989. The retention time of the monomeric AE8 scFv fraction was 87.36 min and this yielded an estimated molecular weight of 28 kDa.

FIG. 8a: Neutravidin pre-concentration analyses. 50 μg/ml solutions of neutravidin were prepared in 10 mM sodium acetate buffers and adjusted with 10% (v/v) acetic acid to pH values 4.0, 4.2, 4.4, 4.6, 4.8, and 5.0. A 10 mM sodium acetate buffer at pH 4.6 was chosen as the immobilisation buffer for neutravidin on the CM5 surface.

FIG. 8b: Immobilisation of 50 μg/ml neutravidin onto the CM5 sensor chip surface. (A) EDC/NHS activation, (B) binding of neutravidin, (C) capping of unreacted groups and (D), regeneration pulses of 5 mM NaOH. A final level of 22,428.4 RUs of covalently attached neutravidin was achieved.

FIG. 8c: The response profile of biotinylated-Neu5Gc-PAA binding to a CM5 chip with immobilised neutravidin. A 100 μg/ml solution of biotinylated-Neu5Gc-PAA in EMS was passed over the chip surface at 10 μl/min for 20 minutes. A final level of 1,398.8 RU of captured biotinylated-Neu5Gc-PAA was achieved.

FIG. 9a: A reference subtracted sensorgram depicting the binding of the AE8 recombinant anti-sialic scFv to Neu5Gc. The AE8 protein was purified by IMAC and a 1 in 100 dilution in HBS was passed over flow cells 1 and 2 of the Neu5Gc sensor chip at 10 μl/min for 7 minutes. The net binding response achieved by AE8 was 1,077.4 RU. The reference subtracted control surface (flow cell 1) consisted of immobilised neutravidin with no biotinylated Neu5Gc-PAA.

FIG. 9b: A Biacore inhibition curve of the anti-sialic binding scFv (AE8). The R/Ro was calculated by dividing the RU response obtained at different Neu5Gc-BSA conjugate concentrations (1000 ng/ml, 500 ng/ml, 250 ng/ml, 125 ng/ml, 62.5 ng/ml and 31.25 ng/ml) by the RU response obtained from the AE8 sample with no Neu5Gc-BSA conjugate. A 4-parameter equation was fitted to the data set using BIAevaluation 4.1 software. Each point in the curve is the mean of three replicate measurements.

FIG. 9c: Curve fitting of monomeric AE8 scFv with BIAevaluation 4.1 using a 1:1 binding model. The fitted curves for each monomeric scFv concentration are represented by the dashed line, whereas the solid lines represent the actual RU change for each sample. The apparent KD was estimated to be 57×10-9 M. The residual plot is a measure of the ‘goodness of fit’.

### DETAILED DESCRIPTION OF THE EMBODIMENTS

### 1.0. New Strategies for Immunisation to Generate Anti-Sialic Acid Antibodies

The generation of anti-carbohydrate antibodies is a notoriously difficult task. Carbohydrate antigens are self-antigens and thus have low antigenic potential. As a consequence, a poor immune response is generated from carbohydrate antigens and the antibody produced is typically a low-affinity immunoglobulin M (IgM).⁷ Conventional hybridoma technologies have been shown to be ineffective at generating high-affinity monoclonal antibodies against a range of carbohydrate structures. In contrast, display technologies such as phage display offer greater potential, as this technology can sometimes generate antibodies against ‘self-antigens’.⁸ Moreover, the complementarity-determining regions (CDRs) of scFv fragments can be targeted and mutagenised to enhance their sensitivity and specificity for particular carbohydrate elements.⁹ The vast majority of anti-carbohydrate antibodies generated have relatively low-affinities and are therefore not suitable for in vitro diagnostics. To overcome the problem of immunological tolerance, a carefully designed protocol was developed for the generation of a suitable immune response to sialic acid. For our study, Gallus domesticus was selected as the animal model. Several reports have suggested that the primary antigen on Neu5Gc, the Hanganutziu-Deicher (HD) antigen, is absent on avian cells. This observation permitted the selection of a novel Neu5Gc-containing immunogen for immunisations. Sialic acid was seen as foreign by the avian immune system and thus generated a strong immune response. For T-cell recognition and the subsequent generation of an immune response, the Neu5Gc monosaccharide was conjugated to a suitable carrier protein. Human Serum Albumin (HSA) was deemed to be appropriate as it facilitated the conjugation of multiple Neu5Gc residues. A second sialic acid protein conjugate was also designed. This conjugate, Bovine Serum Albumin (BSA), was used for phage display screening, recombinant protein screening and the measurement of the avian polyclonal serum response.

### 1.1. Synthesis of the Neu5Gc-BSA and Neu5Gc-HSA Conjugates

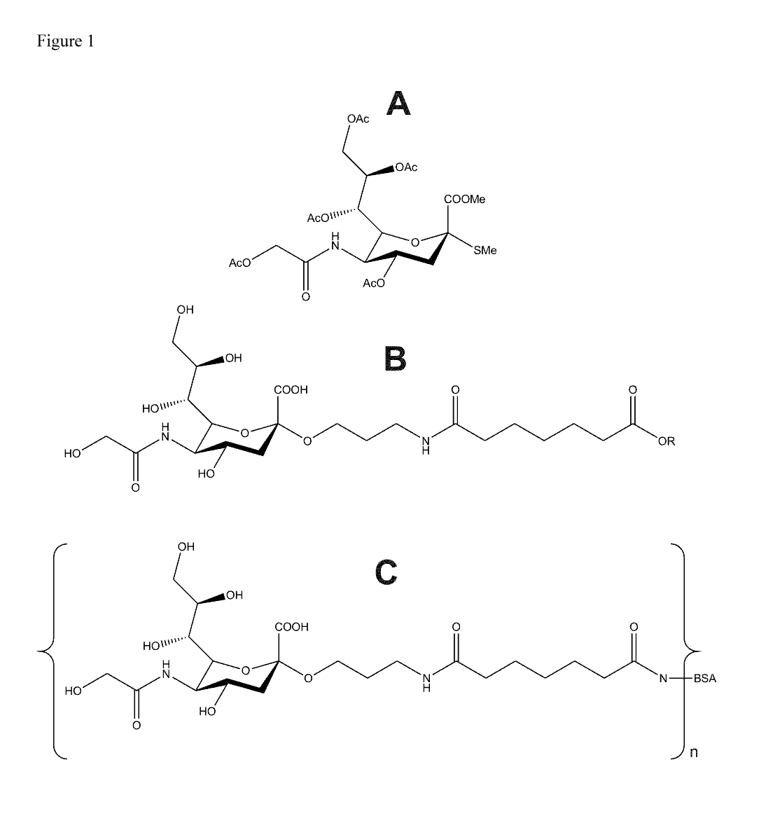

Both the Neu5Gc-BSA and Neu5Gc-HSA conjugates were custom synthesised by Carbohydrate Synthesis, U.K. An overview of the synthesis scheme is given in FIG. 1.

### 1.2. Immunisation of Chickens Using HSA-Neu5Gc

All procedures involving the use of animals were sanctioned by the local ethics committee at Dublin City University (DCU, Dublin, Ireland). In addition, these experiments were approved and licensed by the Irish Department of Health and Children (Dublin, Ireland) and were performed with the highest standards of care. A white male leghorn chicken (aged one month) was injected with 250 μg/ml of the HSA-Neu5Gc conjugate (35 monosaccharide units of Neu5Gc per mole of HSA protein) and an equal volume of Freund's complete adjuvant (FCA). The chicken was injected subcutaneously (200 μl) at four different sites. Following the first injection, the second, third and fourth boosts were given at two, three, and two weekly intervals respectively. For boosting injections, an equal volume of Freund's incomplete adjuvant was used. A bleed was taken after the fourth boost and a serum-based polyclonal response was determined by ELISA. For comparative analysis, a pre-bleed sample (taken from the same host pre-immunisation) was also selected.

### 1.3. Detection of Sialic Acid Antibodies in Polyclonal Avian Serum

1.3.1. Direct ELISA with BSA-Neu5Gc Conjugate

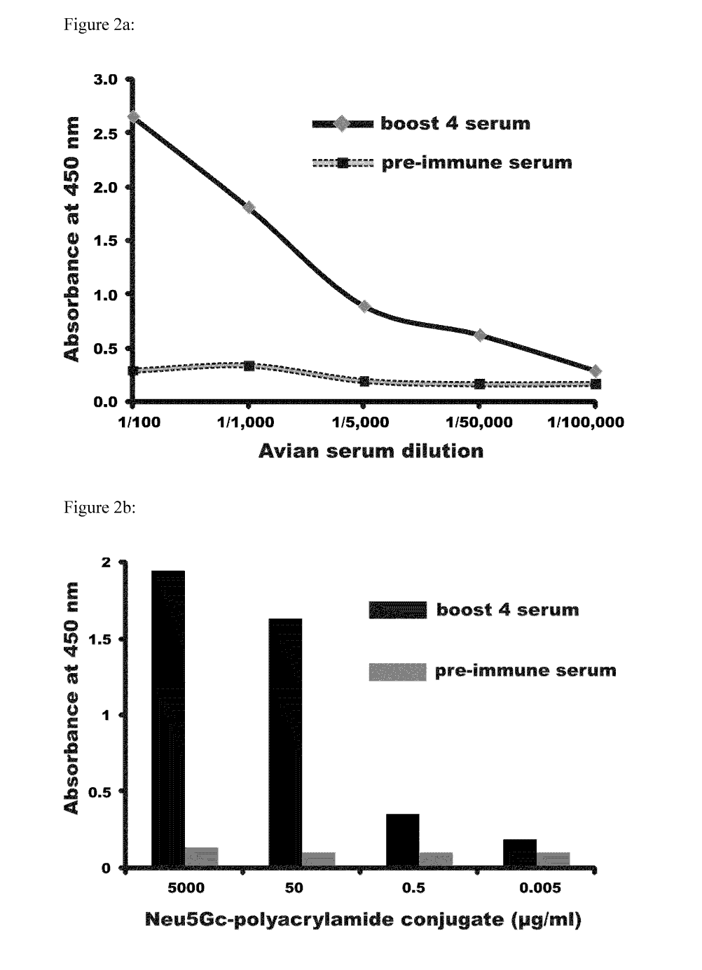

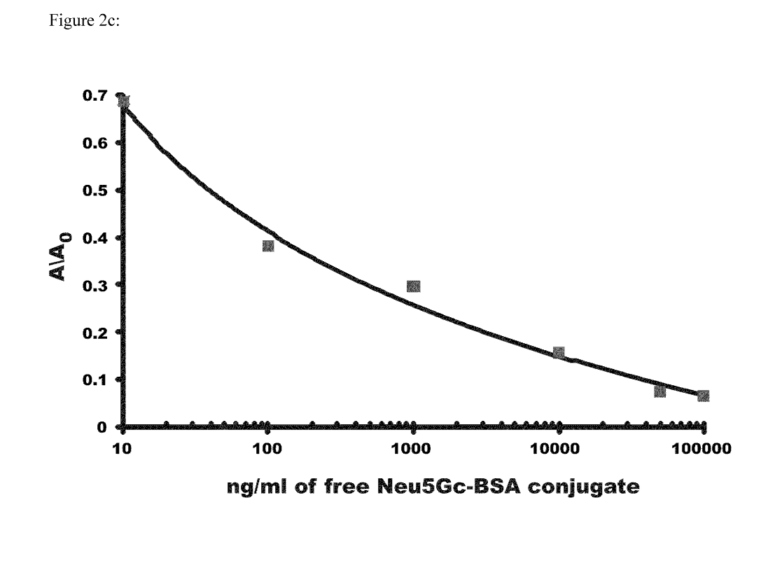

A direct ELISA was used to determine the serum antibody titre from an immunised chicken (FIG. 2a). A Maxisorp plate (Nunc A/S, Denmark) was coated overnight at 4° C. with 5 μg/ml of the BSA-Neu5Gc conjugate (custom synthesised by Carbohydrate Synthesis, U.K.). The plate was blocked with 3% (w/v) BSA in phosphate-buffered saline (PBS (pH 7.2); NaCl 5.84 g/l, Na₂HPO₄ 4.72 g/l and NaH₂PO₄ 2.64 g/l) for 1 hour at 37° C. The plate was washed three times with PBST, pH 7.2 (PBS containing 0.5% (v/v) Tween) followed by three times with 1×PBS, pH 7.2. A series of dilutions ranging from neat to 1 in 1,000,000 of the chicken serum, diluted in 1% (v/v) BSA 1×PBST (pH 7.2), were added to the ELISA plate in triplicate and incubated for 1 hour at 37° C. The plate was washed three times with 1×PBST (pH 7.2) followed by three times with 1×PBS (pH 7.2). 100 μl of rabbit anti-chicken IgY, conjugated with horseradish peroxidase (HRP) (Sigma, U.K., 1:2000 1% (v/v) BSA PBST), was added to the plate and then incubated for 1 hour at 37° C. The plate was washed 3 times with 1×PBST (pH 7.2) and 1×PBS (pH 7.2) and 100 μl of TMB substrate (Sigma-Aldrich, U.K.) solution (2 mg/ml TMB in citrate 0.05M phosphate-citrate buffer, pH 5.0, Sigma-Aldrich, U.K.) was added to each well. The plate was incubated at room temperature to allow chromophore development, after which the reaction was stopped by the addition of 100 μl of 10% (v/v) HCl. The optical density (O.D.) was determined at 450 nm with a Tecan Safire plate reader (Tecan, U.K.).

1.3.2. Direct ELISA with PAA-Neu5Gc Conjugate

To ensure that the avian polyclonal response was directed towards the Neu5Gc component of the conjugate and not the synthetic linker or protein element, the polyclonal serum was also tested against a synthetic carbohydrate that consisted of a multivalent biotinylated polyacrylamide (PAA) polymer that contained 0.2 moles of Neu5Gc per mole of PAA (Glycotech, USA) (FIG. 2b). The serum IgY response against the Neu5Gc antigen was measured by direct ELISA. A 96 well Maxisorp plate was coated overnight at 4° C. with 5 μg/ml of neutravidin (Pierce, U.K.) prepared in coating buffer (1×PBS, pH 7.2). The plate was washed three times with 1×PBST (pH 7.2) and three times with 1×PBS (pH 7.2). 100 μl of biotinylated-PAA-Neu5Gc (25 μg/ml) was added to the plate and incubated for one hour at 37° C. The plate was then blocked with 3% (w/v) BSA solution in 1×PBS (pH 7.2) for 1 hour at 37° C. After washing three times with 1×PBST (pH 7.2) and 1×PBS (pH 7.2), 100 μl of serially-diluted serum (in 1% (v/v) BSA 1×PBST (pH 7.2) blocking buffer) was added to the relevant wells. After 1 hour at 37° C., the plates were washed as before and 100 μl of rabbit anti-chicken IgY conjugated with HRP was added and the plate was then incubated for a further 1 hour. The plate was washed 3 times with 1×PBS (pH 7.2) and 1×PBST (pH 7.2). Subsequently, 100 μl of TMB substrate (Sigma-Aldrich, U.K.) solution (2 mg/ml TMB in citrate 0.05M phosphate-citrate buffer, pH 5.0, Sigma-Aldrich, U.K.) was added to each well. The plate was incubated at room temperature to allow chromophore development, after which the reaction was stopped by the addition of 100 μl of 10% (v/v) HCl. The optical density (O.D.) was determined at 450 nm with a Tecan Safire plate reader (Tecan, U.K.).

1.3.3. Inhibition ELISA with BSA-Neu5Gc Conjugate

A Maxisorp nunc plate was coated overnight at 4° C. with 100 μl of 5 μg/ml of the BSA-Neu5Gc conjugate. The plate was blocked with 3% (w/v) BSA prepared in 1×PBS (pH 7.2) and incubated for 1 hour at 37° C. The plate was washed three times with 1×PBST (pH 7.2) and 1×PBS (pH 7.2). The Neu5Gc-BSA conjugate was added at varying concentrations to a 1:50,000 dilution of avian serum in 1% (v/v) BSA in 1×PBST (pH 7.2). Samples containing no conjugate (A₀) were diluted in 1×PBST (pH 7.2) to ensure the same serum concentration. Sample dilutions were incubated for 2 hours at 37° C. and 100 μl of sample was added to the relevant wells. After a 1 hour incubation at 37° C., the plates were washed three times with 1×PBST (pH 7.2) and 1×PBS (pH 7.2) and 100 μl of rabbit anti-chicken IgY conjugated with HRP was added and the plate was then incubated for 1 hour. The plate was washed three times with 1×PBST (pH 7.2) and 1×PBS (pH 7.2). Subsequently, 100 μl of TMB substrate (Sigma-Aldrich, U.K.) solution (2 mg/ml TMB in citrate 0.05M phosphate-citrate buffer, pH 5.0, Sigma-Aldrich, U.K.) was added to each well. The plate was incubated at room temperature to allow chromophore development, after which the reaction was stopped by the addition of 100 μl of 10% (v/v) HCl. The optical density (O.D.) was determined at 450 nm with a Tecan Safire plate reader (Tecan, U.K.). Results are shown in FIG. 2c.

### 2.0. Generation of Recombinant Anti-Sialic Acid Antibodies

Novel sialic acid binding clones were identified from an avian immune library using the phage display technique. Phage display is a well-established and powerful technique for the discovery and characterisation of antibody fragments (scFv or Fab) that bind to a panel of specific ligands (proteins, carbohydrates or haptens). In this method, antibody fragments are displayed on the outer surface of filamentous phage by inserting short gene fragments in-frame, most commonly into gene III of the phage. This technique couples a polypeptide or peptide of interest to the DNA that encodes it, thus making it possible to select these two characteristics together. The gene III minor coat protein is important for proper phage assembly and for infection by attachment to the pili of Escherichia coli. The gene III fusions are translated into chimeric proteins and phage that display proteins with high binding affinities for the target ligand are readily selected. Affinity can be enriched (matured) through multiple rounds of biopanning, a process that involves binding to reducing concentrations of the immobilised ligand. Phage that are weakly bound, or have low affinity for the antigen are removed by stringent washing steps during biopanning. The high-affinity bound phage are removed from the surface of an immunotube/maxisorb plate by acid or trypsin elution, and amplified through infection of mid-logarithmic growth-phase E. coli cells. Typically, 4 to 6 rounds of panning and amplification are sufficient to select for phage displaying high-affinity antibody fragments.¹⁰ Many publications exist that describe the display of antibody fragments on the surface of the bacteriophage.¹¹ However, the use of this technique to generate anti-carbohydrate antibody fragments is a much less developed area, although some examples of this have been described.¹² However, no publications currently exist that have used an avian host to generate a recombinant antibody fragment (scFv) that can recognise both major forms of sialic acid (Neu5Gc/Neu5Ac) by phage display.

2.1. Isolation and Quantification of Total Cellular RNA from the Spleen and Bone Marrow of an Immunised Leghorn Chicken.

The immunised Leghorn chicken was sacrificed. Both the spleen and the femurs were immediately harvested and processed in a Laminar flow hood (Gelaire BSB 4) that was thoroughly cleaned with 70% (v/v) industrial methylated spirits (IMS, Lennox) and RNaseZAP© (Invitrogen, USA). The bone marrow from the chicken femurs was washed out with 10 mls of chilled TRIzol® reagent (Invitrogen, USA) using a 25 gauge needle and 5 ml syringe. 10 mls of chilled TRIzol® reagent was added to the avian spleen and all samples were fully homogenised using a sterile (autoclaved and baked overnight at 180° C.) homogeniser (Ultra-Turrax model TP 18/10, IKA® Werke GmbH & Co. KG, Germany). The tubes were incubated at room temperature for 5 minutes and centrifuged (eppendorf centrifuge 5810R) at 3500 rpm for 10 minutes at 4° C. The supernatants were carefully removed and transferred to fresh ‘RNase-free’ 50 ml Oakridge tubes (Thermo Fisher Scientific, USA). For each sample, 3 mls of ‘RNase-free’ chloroform (Sigma-Aldrich) were added and tubes were shaken vigorously for 15 seconds, stored at room temperature for 15 minutes and subsequently centrifuged at 17,500 rpm at 4° C. for an additional 15 minutes. Following centrifugation, the mixture separated into a lower phenol-chloroform phase, an interphase, and a colourless upper aqueous phase. The upper aqueous phase, containing the RNA, was carefully removed and transferred to a fresh ‘RNase-free’ 50 ml Oakridge tube. For each sample, 15 mls of propan-2-ol (Sigma-Aldrich) was added and tubes were shaken vigorously for 15 seconds, stored at room temperature for 10 minutes and centrifuged at 17,500 rpm at 4° C. for 30 minutes. RNA precipitated as a white gel-like pellet on the bottom and side of the tube. The supernatant was removed and the pellet was washed with 30 mls of 75% (v/v) ethanol (Sigma-Aldrich) and centrifuged at 17,500 rpm at 4° C. for 10 minutes. This step was repeated and after removal of the supernatant, the RNA pellet was allowed to air dry for 5 minutes. The pellet was then resuspended in 250 μl of ‘RNase-free’ water (Sigma-Aldrich). The RNA concentrations were determined by spectrophotometric measurement at 260 nm with a NanoDrop™ spectrophotometer ND-1000 (Thermo Fisher Scientific, USA). The purity of the RNA preparation was assessed by measuring the ratio of absorbance at 260 nm and 280 nm. Furthermore, sample purity was assessed by analysis on a 1% (w/v) agarose gel. An aliquot of freshly isolated RNA was used for cDNA synthesis. The remaining RNA solution was precipitated at −20° C. with 1/10 the volume of ‘RNase-free’ sodium acetate pH 5.2 (Sigma-Aldrich) and 2 times the total sample volume of 100% (v/v) ethanol. To enhance RNA precipitation, nuclease-free Glycogen (Fermentas) was added at a final concentration of 1 μg/ul.

2.1.1. Reverse Transcription of Total RNA to cDNA.

The SuperScript™ III First-Strand Synthesis System for RT-PCR, (Invitrogen, USA) was used to generate first strand-cDNA from 5 μg of total RNA using oligo dT₂₀ priming. All reactions were kept on ice at all times. Two 20× master mixes, hereafter referred to as 1 and 2, were made using the recipe below. 25 μls of reaction mix 1 were added to 8 tubes that were subsequently incubated at 65° C. for 5 minutes (Biometra TGRADIENT PCR machine) and placed on ice for 1 minute. 25 μls of reaction mix 2 were added to the same 8 tubes, the tubes were further incubated at 50° C. for 50 minutes and the cDNA reaction was terminated by incubation at 85° C. for 5 minutes. The samples were spun briefly and 1 μA of RNase H was added to each tube. The tubes were then incubated at 37° C. for 20 minutes, after which the cDNA was pooled, aliquoted and stored at −20° C. To assess cDNA quality samples were run on a 1% (w/v) agarose gel.

| | Stock Concentration | Volume per 1 RxN |
|---|---|---|
| Mix 1-Components | | |
| RNA | X μl (to give 5 μg) | X μl (to give 5 μg) |
| Oligo-dt | 50 μM | 1 μl |
| dNTPs | 10 mM | 1 μl |
| Sterile H₂O | | X μl |
| Total Volume | | 10 μl |
| Mix 2-Components | | |
| RT Buffer | 10× | 2 μl |
| MgCl₂ | 25 mM | 4 μl |
| DTT | 10 mM | 2 μl |
| RNase OUT | 40 U/μl | 1 μl |
| Superscript III RT | 200 U/μl | 1 μl |
| Total Volume | | 10 μl |

### 2.2. PCR Primers and Conditions Used for the Construction of the Avian Library

The following sets of oligonucleotides were used to generate a chicken scFv library with a short linker from both the bone marrow and spleen. All primers were high purity, salt free and were purchased from Eurofins MWG Operon (Ebersberg, Germany).

### Variable Heavy Chain (V_{H}) Primers

CSCHo-F (sense), Short Linker
(SEQ ID NO: 1)
5′ GGT CAG TCC TCT AGA TCT TCC GCC GTG AC GTT GGA
CGA G 3′
CSCG-B (reverse)
(SEQ ID NO: 2)
5′ CTG GCC GGC CTG GCC ACT AGT GGA GGA GAC GAT GAC
TTC GGT CC 3′

### Variable Light Chain (V_{L}) Primers

CSCVK (sense)
(SEQ ID NO: 3)
5′ GTG GCC CAG GCG GCC CTG ACT CAG CCG TCC TCG GTG
TC 3′
CKJo-B (reverse)
(SEQ ID NO: 4)
5′ GGA AGA TCT AGA GGA CTG ACC TAG GAC GGT CAG
G 3′

### Overlap Extension Primers

CSCHo-F (sense)
(SEQ ID NO: 5)
5′ GAG GAG GAG GAG GAG GAG GTG GCC CAG GCG GCC CTG
ACT CAG 3′
CSC-B (reverse)
(SEQ ID NO: 6)
5′ GAG GAG GAG GAG GAG GAG GAG CTG GCC GGC CTG GCC
ACT AGT GGA GG 3′

For V_{H} and V_{L}, gene amplification, a 100 μl PCR reaction contained the following: 1 μl of cDNA, 60 pMole of CSCHo-F and CSCG-B, 5×PCR Buffer (Promega, USA), 1.5 mM MgCl₂ (Promega, USA), 200 μM dNTPs (Promega, USA), and 0.5 μl GoTaq® DNA Polymerase (Promega, USA).

For V_{L}, gene amplification the PCR reaction components were the same except that 60 pmole of CSCVK and CKJo-B were used in place of the V_{H} primers. The Hybaid Thermal Cycler (Thermo Px2, Thermo Fisher Scientific, USA) was used for all PCR reactions. Touchdown PCR was performed with the following cycling conditions: 4 minutes at 94° C. (initial denaturation), followed by 30 cycles of 15 sec at 94° C. (denaturation), 30 sec at 60° C. (annealing)—the annealing temperature of each cycle was decreased by 0.1° C., 45 sec at 72° C. (extension) and the reaction was terminated after 5 minutes at 72° C. (final extension). The resulting PCR products were run analysed a 1% (w/v) agarose gel and purified with the Wizard® SV Gel and PCR Clean-Up System (Promega, USA) according to the manufacturer's instructions. The V_{H} and V_{L}, purified fragments were joined with a glycine-serine linker (Gly₄Ser)₃ using splice overlapping extension (SOE) PCR. The resulting PCR product was an amplicon approximately 750 bp in length. For SOE-PCR, a 100 μl PCR reaction contained the following: 100 ng of the V_{L}, and V_{H} purified products, 60 pMolar of CSC-F and CSC-B, 10×PCR Buffer (Invitrogen, USA), 1.5 mM MgSO₄ (Invitrogen, USA), 200 μM dNTPs and 1 μl Platinum® Taq DNA Polymerase (Invitrogen, USA).

PCR was performed with the following cycling conditions: 5 minutes at 94° C. (initial denaturation), followed by 30 cycles of 30 sec at 94° C. (denaturation), 30 sec at 57° C. (annealing), 1 minutes at 72° C. (extension) and the reaction was terminated after 10 minutes at 72° C. (final extension). The resulting PCR products were run on a 1% (w/v) agarose gel and purified with the Wizard® SV Gel and PCR Clean-Up System according to the manufacturer's instructions.

2.2.1. SOE-PCR Restriction Digestion and Ligation into pComb3XSS Vector for Phage Display.

The scFv fragment and the cloning vector pComb3XSS were digestion with the Sfi 1 restriction enzyme. Prior to digestion, the vector and scFv DNA concentrations were determined by absorbance measurement at 260 nm with the NanoDrop™ ND1000 spectrophotometer. For scFv digestion, a 100 μl reaction contained the following: 12 μg of gel-purified short linker scFv, 200 units of Sfi 1 (New England Biolabs, USA), 10×NEBuffer 2 (New England Biolabs, USA) and 10×BSA (New England Biolabs, USA). The pComb3XSS 100 μl digestion reaction contained the following: 40 μg of gel-purified vector, 240 units of Sfi1, 10×NEBuffer 2 and 10×BSA. The digestion of purified insert (scFv) and vector (pComb3XSS) was performed for 5 hours at 50° C.

Following digestion, the cut pComb3XSS vector and the scFv fragment were purified from a 1% (w/v) agarose gel using the Wizard® SV Gel and PCR Clean-Up System and DNA quantification was determined at 260 nm using the NanoDrop™ ND1000 spectrophotometer. The ligation of the scFv fragment with the pComb3XSS vector (ratio of vector to insert 2:1) was performed using T4 DNA ligase (New England Biolabs, USA) overnight at room temperature. The 200 μl ligation mixture contained the following: 1.4 μg of gel-purified and stuffer free pComb3XSS vector, 700 ng of gel-purified scFv, 5× ligase Buffer, and 200 units of T4 DNA ligase. After ligation, the solution was precipitated at −20° C. with 1/10 the volume of ‘RNase-free’ sodium acetate (pH 5.2), 2 times the volume of 100% (v/v) ethanol and 41 of Pellet Paint® NF co-precipitant (Merck, U.K.) After overnight precipitation, the sample was centrifuged at 14000 rpm for 20 minutes at 4° C. and the pellet was washed with 70% (v/v) ice-cold ethanol. The mixture was centrifuged at 14000 rpm for 10 minutes at 4° C. and the pellet was resuspended in 5 μl of molecular grade water (Sigma-Aldrich, USA).

2.3. Electro-Transformation of XL-1 Blue E. coli Cells with scFv-Containing Plasmid.

Commercially available electrocompetent XL-1 blue E. coli cells (Stratagene, USA) were transformed with the ligated scFv vector construct. This was achieved using a Gene Pulser Xcell electroporation system (Bio-Rad Laboratories, USA) with the controls set at 25 μF, 1.25 kV and the Pulse Controller at 200Ω. The E. coli cells (50 μl) were thawed on ice. The ligated product (2 μl) was added to the cells, mixed, left to incubate for 30 seconds and immediately transferred to an ice-cold 0.2 cm electroporation cuvette (Bio-Rad Laboratories, USA). The cuvette was tapped so that the suspension was at the base and was placed in the ShockPod and pulsed once. The cuvette was quickly removed from the chamber and 1 ml of SOC medium (SOB medium containing 20 mM glucose; SOB medium contains: tryptone 20 g/l, yeast extract 5 g/l, NaCl 0.5 g/1, 186 mg/l KCl, 10 mM MgCl₂ and 10 mM MgSO₄ (Sigma-Aldrich, USA)) was added immediately to the cuvette. The cells were quickly but gently resuspended with a sterile Pasteur pipette. The 1 ml suspension was transferred to a 20 ml sterile universal container containing 2 mls of SOC media. To facilitate recovery of the cells, the universal container was shaken for 1 hour at 250 rpm at 37° C. The pComb3XSS transformants were plated on TYE plates (tryptone 10 g/l, yeast extract 5 g/l, NaCl 8 g/l and bacto agar 15 g/l (Sigma-Aldrich, USA)), supplemented with 100 μg/ml carbenicillin (Sigma-Aldrich, USA) and 1% (v/v) glucose (Sigma-Aldrich, USA). Untransformed XL-1 blue E. coli cells (negative control) were plated out in parallel on agar plates with 100 μg/ml carbenicillin and 1% (v/v) glucose. The plates were incubated overnight at 37° C. The pComb3XSS transformant colonies were scraped off the plates and used as library stocks. These cells were suspended in 20% (v/v) glycerol, snap frozen in liquid nitrogen and stored at −80° C.

2.4. Rescue of scFv-Displaying Phage.

The anti-sialic acid spleen and bone marrow libraries were propagated using 2×600 μl inoculums of cells (from the frozen glycerol stocks) into 2×600 mls cultures of 2×TY (tryptone 12 g/l; yeast extract 10 g/l; NaCl 5 g/l; final (pH 7.2) containing 100 μg/ml carbenicillin and 2% (w/v) glucose. These libraries were propagated at 200 rpm and 37° C. until mid-exponential phase of growth (O.D. ˜0.600 @ 600 nm). The cultures were spun down at 4000 rpm at 4° C. for 10 minutes. The pellets were resuspended in fresh 2×TY media (600 mls) containing 100 μg/ml carbenicillin and 1×10¹¹ plaque-forming units (pfu)/ml of M13KO7 helper phage (New England Biolabs, USA). The cultures were incubated at 37° C. for 30 minutes without agitation after which time they were propagated at 200 rpm and 37° C. for 2 hours. Subsequently, carbenicillin (100 μg/ml) and kanamycin (50 μg/ml, Sigma-Aldrich, USA) were added and the cultures were grown overnight (200 rpm, 30° C.). The cultures were centrifuged at 4000 rpm for 15 minutes at 4° C. and the supernatants transferred to clean sterile 250 ml Sorval centrifuge tubes (Thermo Fisher Scientific, USA). The phage particles were precipitated by the addition of polyethyleneglycol 8000 (to 4% (w/v)) and NaCl (to 3% (w/v)) (Sigma-Aldrich, USA). The PEG-NaCl solution was dissolved by shaking at 200 rpm for 10 minutes at 37° C. The 250 ml centrifuge tube was placed on ice for 1 hour at 4° C. and centrifuged at 8000 rpm for 25 minutes at 4° C. The phage/bacterial pellet was resuspended in 2 ml Tris-EDTA (Sigma-Aldrich, USA) buffer in 2% (w/v) BSA (Sigma-Aldrich, USA) solution. After the phage pellet was transferred to 1.5 ml sterile centrifuge tubes, it was centrifuged at 14000 rpm for 5 minutes at 4° C. The supernatant containing the phage scFv was placed on ice and stored at 4° C.

2.4.1 Selection of Sialic Acid Binding Phage scFv Fragment by Panning with Immobilised Neu5Gc-BSA.

Maxisorp Immuno-tubes™ (Thermo Fisher Scientific, USA) were coated overnight at 4° C. with 500 μl of 100 μg/ml Neu5Gc-BSA conjugate. The tubes were blocked with 4 mls of 3% (w/v) BSA in 1×PBS (pH 7.2) for 2 hours at room temperature. The blocking solution was removed and 500 μl of rescued phage was added and incubated on a tube roller-mixer SRT1 (Bibby Scientific, U.K.) for 2 hours at room temperature. The solution was removed and non-binding phage were discarded by washing three times with 1×PBST (pH 7.2) and 1×PBS (pH 7.2). Excess PBS was discarded and bound phage particles were eluted with 500 μl of 10 mg/ml type II porcine trypsin (Sigma-Aldrich) in 1×PBS (pH 7.2) solution. The Immuno-tubes™ were then incubated at 37° C. for 30 minutes Half the eluted phage particles (250 μl) were stored at 4° C. The other 250 μl of phage were infected into 2 mls of mid-exponential phase XL-1 blue E. coli cells. After a static 30 minute incubation at 37° C., 20 μl of culture was removed and serial diluted (10⁻¹-10⁻¹²) in 2×TY media. Serial dilutions (10⁻⁸-10⁻⁴) were spread on 2×TY agar plates containing 100 μg/ml carbenicillin and incubated overnight at 37° C. The remaining culture was propagated at 200 rpm for 1 hour at 37° C. Cells were harvested by centrifugation at 4000 rpm for 10 minutes at 4° C. Library plates were prepared by resuspending the cell pellet in 600 μl of fresh 2×TY media and by spread-plating on TYE plates containing 1% (w/v) glucose and 100 μg/ml carbenicillin. Plates were incubated overnight at 37° C.

Input titres were performed by infecting mid-exponential growth phase XL-1 blue E. coli cells (180 μl) with 20 μl of precipitated phage (stored at 4° C.) for 15 minutes at 37° C. and serial dilutions were performed (10⁻¹-10⁻¹²) and spread on 2×TY agar plates containing 100 μg/ml carbenicillin and incubated overnight at 37° C. In the subsequent rounds of biopanning (2, 3, 4, and 5) the bone marrow and spleen libraries, only 100 mls of 2×TY medium was used for cell propagation. In addition, two different phage elution strategies were followed namely (A) competitive elution and (B) standard tyrpsin elution. For the standard trypsin elution method, the Neu5Gc coating concentrations of the Immuno-tubes™ were reduced in rounds 3, 4, and 5 of biopanning to a concentration of 30 μg/ml, 20 μg/ml, and 10 μg/ml, respectively. In addition, the washing of the Immuno-tubes™ was increased as follows: round three, 6 times 1×PBST (pH 7.2) and 1×PBS (pH 7.2), round four, 9 times 1×PBST (pH 7.2) and 1×PBS (pH 7.2) and round five, 12 times 1×PB ST (pH 7.2) and 1×PBS (pH 7.2). For competitive elution the Neu5Gc-BSA and Neu5Gc-PAA conjugates were added to the Immuno-tubes™ and incubated overnight at 4° C.

The next day the eluted phage were infected into 2 mls of mid-exponential phase XL-1 blue E. coli cells. After a static 30 minute incubation at 37° C., 20 μl of culture was removed and serial diluted (10⁻¹-10⁻¹²) in 2×TY media. Serial dilutions (10⁻⁸-10⁻⁴) were spread on 2×TY agar plates containing 100 μg/ml carbenicillin and incubated overnight at 37° C. The remaining culture was propagated at 200 rpm for 1 hour at 37° C. Cells were harvested by centrifugation at 4000 rpm for 10 minutes at 4° C. Library plates were prepared by resuspending the cell pellet in 600 μl of fresh 2×TY media and by spread-plating on TYE plates containing 1% (w/v) glucose and 100 μg/ml carbenicillin. Plates were incubated overnight at 37° C. For each successive round of biopanning using competitive elution, 500 μl of the Neu5Gc-BSA and Neu5Gc-PAA conjugates in 1% (w/v) BSA 1×PBST (pH 7.2) were added to the Immuno-tubes™ at the following concentrations: round 2, 500 μg/ml of Neu5Gc-BSA and 40 μg/ml Neu5Gc-PAA, round 3, 300 μg/ml of Neu5Gc-BSA and 20 μg/ml Neu5Gc-PAA, round 4, 200 μg/ml of Neu5Gc-BSA and 20 μg/ml Neu5Gc-PAA, and round 5, 100 μg/ml of Neu5Gc-BSA and 10 μg/ml Neu5Gc-PAA. The Immuno-tubes™ coating concentration (50 μg/ml) and washing (three times 1×PBST (pH 7.2) and 1×PBS (pH 7.2) was kept the same for all rounds of competitive elution panning.

### 2.5. Polyclonal Phage ELISA Analysis.

For the determination of affinity maturation, a polyclonal phage ELISA was performed. A 96-well plate was coated overnight at 4° C. with 100 μl of 10 μg/ml Neu5Gc-BSA. The plate was then blocked for 1 hour at 37° C. with 3% (w/v) BSA in 1×PBS (pH 7.2). After blocking, the plate was washed three times with 1×PBST (pH 7.2) and 1×PBS (pH 7.2). 100 μl of phage particles from each round of panning (diluted 1:10 in 1% (w/v) BSA 1×PBST, pH 7.2) were assayed in triplicate. Plates were washed three times with 1×PBST (pH 7.2) and 1×PBS (pH 7.2) and 100 μl of a 1:5000 dilution of HRP-conjugated mouse anti-M13 monoclonal antibody (GE Healthcare, U.K.) in 1% (w/v) BSA 1×PBST (pH 7.2) was added for 1 hour at room temperature. The plate was washed three times with 1×PBST (pH 7.2) and 1×PBS (pH 7.2). Subsequently, 100 μl of TMB substrate (Sigma-Aldrich, U.K.) solution (2 mg/ml TMB in citrate 0.05M phosphate-citrate buffer, pH 5.0, Sigma-Aldrich, U.K.) was added to each well. The plate was incubated at room temperature to allow chromophore development, after which the reaction was stopped by the addition of 100 μl of 10% (v/v) HCl. The optical density (O.D.) was determined at 450 nm with a Tecan Safire plate reader (Tecan, U.K.). Good affinity maturation for both libraries was seen when assayed on Neu5Gc-BSA.

### 2.6. Production and Analysis of Soluble Antibody Fragments.

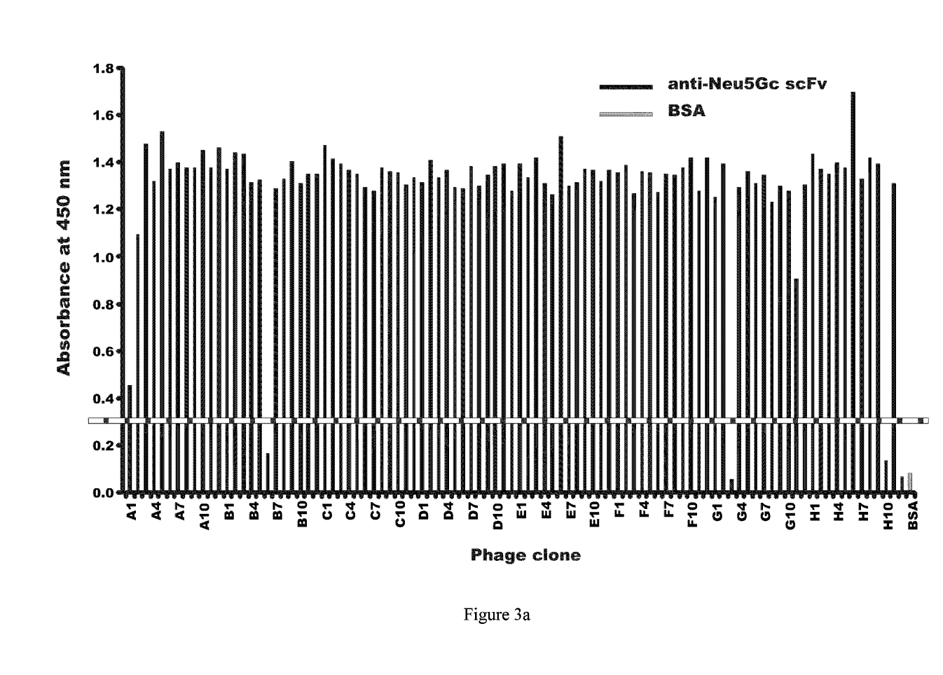

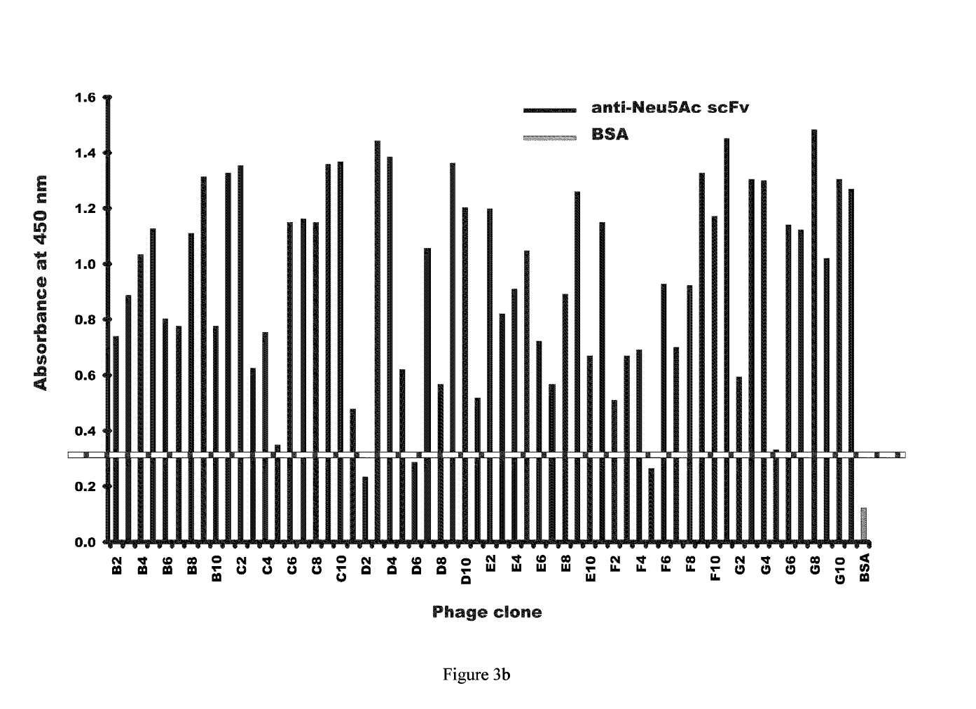

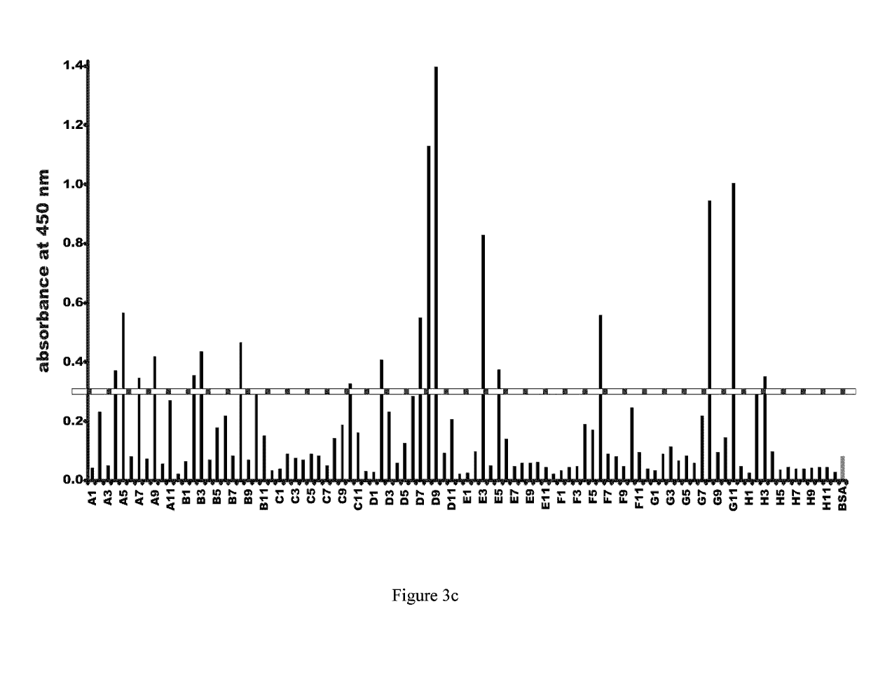

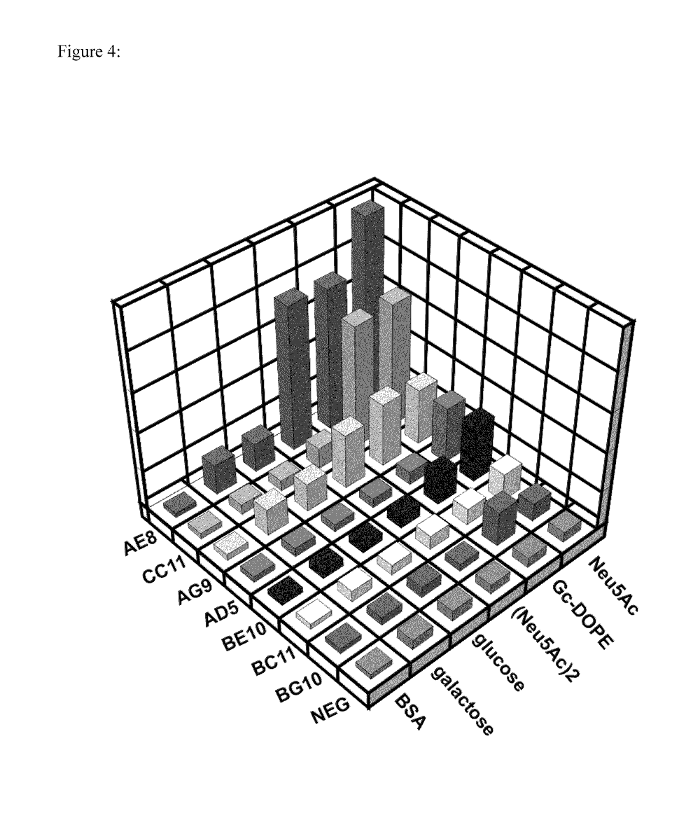

Antibody fragments without the pIII protein were produced by infecting phagemid DNA from rounds 3 and 4 of panning into E. coli TOP 10F′ cells (Stratagene, USA) at mid-logarithmic growth phase. After incubation for 30 minutes at 37° C., serial dilutions were prepared in 2×TY (10⁻² to 10⁻¹⁰), and plated on TYE plates containing 1% (w/v) glucose and 100 μg/ml carbenicillin. Single colonies were inoculated into individual wells of a 96-well ELISA plate containing 200 μl of 2×TY in the presence of carbenicillin (100 μg/ml) and glucose (1.0% (w/v)). After an overnight incubation at 37° C., master plates of the original clones were prepared by adding glycerol (20% (w/v)) and storing at −80° C. These plates were used as a backup stock for each putative clone of interest. Twenty μl from the overnight subculture plates were inoculated into fresh 2×TY media (180 μl) containing 1×505 medium (0.5% (v/v) glycerol, 0.05% (v/v) glucose final concentration), 1 mM MgSO₄ and 100 μg/ml carbenicillin. The sterile 96 well plates were propagated at 37° C. at 180 rpm until a cell density of ˜0.600 was achieved. A final concentration of 1 mM Isopropyl-β-D-thiogalactoside (IPTG) was added to each individual well and the plates were induced overnight at 180 rpm at 30° C. The overnight cultures were frozen at −80° C. The periplasmic scFv was extracted from the cells by three cycles of freeze-thaw. Cell extracts were cleared by centrifugation (4000 rpm, 10 minutes) and the lysates were diluted 1:5 in 1% (w/v) BSA 1×PBS (pH 7.2). ELISA-based analysis was performed as follows. A 96-well plate was coated overnight at 4° C. with 100 μl of 10 μg/ml Neu5Gc-BSA. The plate was then blocked for 1 hour at 37° C. with 3% (w/v) BSA in 1×PBS (pH 7.2). After blocking, the plate was washed three times with 1×PBST (pH 7.2) and 1×PBS (pH 7.2). 100 μl of the periplasmic scFv cell extracts (diluted 1:5 in 1% (w/v) BSA 1×PBST, pH 7.2) were assayed in triplicate. Plates were washed three times with 1×PBST (pH 7.2) and 1×PBS (pH 7.2) and 100 μl of a 1:2000 dilution (1% (w/v) BSA 1×PBST (pH 7.2) of rat anti-HA monoclonal antibody conjugated with peroxidase (Roche Diagnostics, USA) was added for 1 hour at 37° C. The plate was washed three times with 1×PBST (pH 7.2) and 1×PBS (pH 7.2). Subsequently, 100 μl of TMB substrate (Sigma-Aldrich, U.K.) solution (2 mg/ml TMB in citrate 0.05M phosphate-citrate buffer, pH 5.0, Sigma-Aldrich, U.K.) was added to each well. The plate was incubated at room temperature to allow chromophore development, after which the reaction was stopped by the addition of 100 μl of 10% (v/v) HCl. The optical density (O.D.) was determined at 450 nm with a Tecan Safire plate reader (Tecan, U.K.). Results for competitively-eluted and trypsin eluted scFvs are shown in FIGS. 3a and 3b, respectively.

### 2.7. Assessing the Ability of the Soluble Anti-Sialic Clones to Recognise Neu5Gc in the Context of a Polyacrylamide Backbone.

In order to identify scFvs that could not only bind Neu5Gc-BSA but also recognise this monosaccharide in the context of an alternative backbone, positive clones identified from monoclonal scFv ELISA-analysis, were tested against Neu5Gc-PAA. A 96 well nunc maxisorb plate was coated overnight at 4° C. with 5 μg/ml of neutravidin in coating buffer 1×PBS (pH 7.2). The plate was washed three times with 1×PBS (pH 7.2) and 1×PBST (pH 7.2). 100 μl of biotinylated-PAA-Neu5Gc (25 μg/ml) was added and the plate was incubated for one hour at 37° C. The plate was blocked with 3% (w/v) BSA solution in 1×PBS (pH 7.2) for 1 hour at 37° C. After washing three times with 1×PBS (pH 7.2) and 1×PBST (pH 7.2), 100 μl of soluble-expressed scFv (diluted 1:5 in 1% (w/v) BSA 1×PBST, pH 7.2) were added to the plate. After a 1 hour incubation at 37° C., the plate was washed three times with 1×PBS (pH 7.2) and 1×PBST (pH 7.2) and 100 μl of 1:2000 dilution (1% (w/v) BSA 1×PBST (pH 7.2)) of an anti-HA rat monoclonal antibody conjugated with peroxidase was added. The plate was incubated for 1 hour, washed 3 times with 1×PBS (pH 7.2) and 1×PBST (pH 7.2). Subsequently, 100 μl of TMB substrate (Sigma-Aldrich, U.K.) solution (2 mg/ml TMB in citrate 0.05M phosphate-citrate buffer, pH 5.0, Sigma-Aldrich, U.K.) was added to each well. The plate was incubated at room temperature to allow chromophore development, after which the reaction was stopped by the addition of 100 μl of 10% (v/v) HCl. The optical density (O.D.) was determined at 450 nm with a Tecan Safire plate reader (Tecan, U.K.). Results for this assay are illustrated in FIG. 3c.

2.7.1. Cross Reaction-Analysis of the Soluble Anti-Neu5Gc Clones with Other Mono and Disaccharides.

The capacity of the anti-Neu5Gc clones to cross-react with other carbohydrate elements was assessed by analysis against the following structures: Neu5Ac-PAA, (Neu5Ac)₂-PAA, Neu5Gc-DOPE, glucose-PAA and galactose-PAA (FIG. 4). A 96 well nunc maxisorb plate was coated overnight at 4° C. with 5 μg/ml of neutravidin in coating buffer 1×PBS (pH 7.2). The plate was washed with three times with 1×PBS (pH 7.2) and 1×PBST (pH 7.2). 100 μl of biotinylated-PAA-conjugate (Neu5Ac-PAA, (Neu5Ac)₂-PAA, Neu5Gc-DOPE, glucose-PAA and galactose-PAA) at 25 μg/ml was added and the plate was incubated for one hour at 37° C. The plate was blocked with 3% (w/v) BSA solution in 1×PBS (pH 7.2) for 1 hour at 37° C. After washing three times with 1×PBS (pH 7.2) and 1×PBST (pH 7.2), 100 μl of soluble-expressed scFv (diluted 1:5 in 1% (w/v) BSA 1×PBST, pH 7.2) were added to the plate. After a 1 hour incubation at 37° C., the plate was washed three times with 1×PBS (pH 7.2) and 1×PBST (pH 7.2) and 100 μl of 1:2000 dilution (1% (w/v) BSA 1×PBST (pH 7.2)) of an anti-HA rat monoclonal antibody conjugated with peroxidase was added. The plate was incubated for 1 hour, washed 3 times with 1×PBS (pH 7.2) and 1×PBST (pH 7.2). Subsequently, 100 μl of TMB substrate (Sigma-Aldrich, U.K.) solution (2 mg/ml TMB in citrate 0.05M phosphate-citrate buffer, pH 5.0, Sigma-Aldrich, U.K.) was added to each well. The plate was incubated at room temperature to allow chromophore development, after which the reaction was stopped by the addition of 100 μl of 10% (v/v) HCl. The optical density (O.D.) was determined at 450 nm with a Tecan Safire plate reader (Tecan, U.K.).

### 2.8. Sequence Analysis of the Anti-Neu5Gc/Neu5Ac Binding Clones

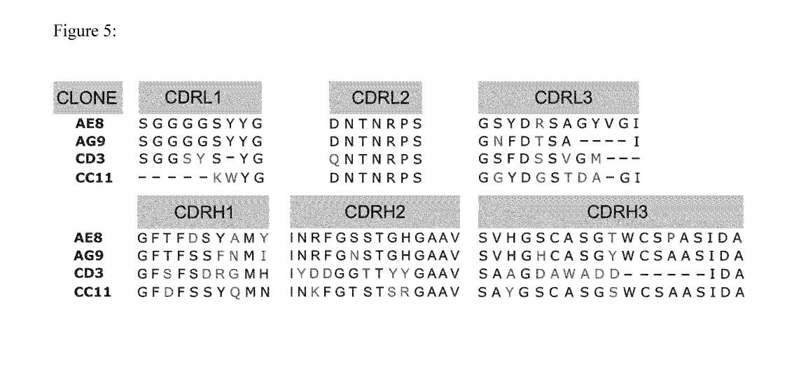

To ensure the fidelity of the sequence data, three different samples (stab culture, plasmid prep and unpurified PCR products) of the same clone were sent for sequencing. Double stranded DNA sequencing of all clones was performed by Eurofins MWG Operon (Ebersberg, Germany). A panel of anti-sialic acid clones were grown in 1.5 ml eppendorf stab cultures. In addition, purified plasmid was obtained from each clone using the Wizard® Plus SV Minipreps DNA Purification System in accordance with the manufacturer's instructions. Furthermore, the scFv gene fragment was also amplified using colony pick PCR. For colony pick PCR, a 50 μl PCR reaction contained the following: 2 μl of an overnight culture, 60 pmole of CSC-F and CSC-B, 5×PCR Buffer, 1.5 mM MgCl₂, 200 μM dNTPs and 0.25 μl GoTaq® DNA Polymerase. Touchdown PCR was performed with the following cycling conditions: 10 minutes at 94° C. (initial denaturation), followed by 30 cycles of 30 sec at 94° C. (denaturation), 30 sec at 56° C. (annealing)—the annealing temperature of each cycle was decreased by 0.1° C., 1 minute at 72° C. (extension) and the reaction was terminated after 10 minutes at 72° C. (final extension). The sequences of the CDLR1, CDLR2, CDLR3, CDHR1, CDHR2 and CDHR3 regions for the AE8, AG9, CD3, and CC1 clones are shown in FIG. 5. The full sequences of the variable heavy and light chains of the clones, and the sequences of the full clones (including the spacer which is underlined) are provided below:

AE8
Variable Light Chain
(SEQ ID NO: 22)
GGTVKITCSGGGGSYYGWFQQKSPGSAPVTVIYDNTNRPSNIPSRFSGSL
SGSTNTLTITGVQAEDEAVYYCGSYDRSAGYVGIFGAGTTLTVL
Variable Heavy Chain
(SEQ ID NO: 23)
AVTLDESGGGLQTPGGGLSLVCKASGFTFDSYAMYWVRQAPGKGLEWVAS
INRFGSSTGHGAAVKGRATISRDNGQSTLGAYPYDVPDYAS
scFv
(SEQ ID NO: 30)
GGTVKITCSGGGGSYYGWFQQKSPGSAPVTVIYDNTNRPSNIPSRFSGSL
SGSTNTLTITGVQAEDEAVYYCGSYDRSAGYVGIFGAGTTLTVLGQSSRS
SAVTLDESGGGLQTPGGGLSLVCKASGFTFDSYAMYWVRQAPGKGLEWVA
SINRFGSSTGHGAAVKGRATISRDNGQSTLGAYPYDVPDYAS
AE9
Variable Light Chain
(SEQ ID NO: 24)
GGTVKITCSGGGGSYYGWFQQKSPGSAPVTVIYDNTNRPSNIPSRFSGSK
SGSTGTLTITVQAEDEAVYYCGNFDTSAIFGAGTTLTVL
Variable Heavy Chain
(SEQ ID NO: 25)
AVTLDESGGGLQTPGGALSLICKASGFTFSSFNMIWVRQAPGKGLEFVGS
INRFGNSTGHGAAVKGRVTISRDDGQSTVRLQLNNLRAEDTGTYFCAKSV
HGHCASGYWCSAASIDAWGHGTEVIVSSTSGQAGQHHHHHH GAYPYDVP
DYAS
scFv
(SEQ ID NO: 31)
GGTVKITCSGGGGSYYGWFQQKSPGSAPVTVIYDNTNRPSNIPSRFSGSK
SGSTGTLTITVQAEDEAVYYCGNFDTSAIFGAGTTLTVLGQSSRSSAVTL
DESGGGLQTPGGALSLICKASGFTFSSFNMIWVRQAPGKGLEFVGSINRF
GNSTGHGAAVKGRVTISRDDGQSTVRLQLNNLRAEDTGTYFCAKSVHGHC
ASGYWCSAASIDAWGHGTEVIVSSTSGQAGQHHHHHHGAYPYDVPDYAS
CD3
Variable Light Chain
(SEQ ID NO: 26)
GGTVEITCSGGSYSYGWYQQKSPGSAPVTVIYQNTNRPSDIPSRFSGSKS
GSTGTLTITGVRAEDEAVYYCGSFDSSVGMFGAGTTLTVL
Variable Heavy Chain
(SEQ ID NO: 27)
AVTLDESEGGLQTPGGALSLVCKASGFSFSDRGMHWVRQAPGKGLEYVAG
IYDDGGTTYYGAAVKGRASITRDNGQSAVRLQLNNLRAEDTATYYCAKSA
AGDAWGADDIDAWGHGTEVIVSSTSGQAGQHHHHHHGAYPYDVPDYAS
scFv
(SEQ ID NO: 32)
GGTVEITCSGGSYSYGWYQQKSPGSAPVTVIYQNTNRPSDIPSRFSGSKS
GSTGTLTITGVRAEDEAVYYCGSFDSSVGMFGAGTTLTVLGQSSRSSAVT
LDESEGGLQTPGGALSLVCKASGFSFSDRGMHWVRQAPGKGLEYVAGIYD
DGGTTYYGAAVKGRASITRDNGQSAVRLQLNNLRAEDTATYYCAKSAAGD
AWGADDIDAWGHGTEVIVSSTSGQAGQHHHHHHGAYPYDVPDYAS
CC11
Variable Light Chain
(SEQ ID NO: 28)
KWYGWYQQKAPGSAPVTLIYDNTNRPSDIPSRFSGSASGSTATLTITGVQ
VEDEAVYFGGYDGSTDAGIFGAGTTLTVL
Variable heavy Chain
(SEQ ID NO: 29)
AVTLDESGGGLQTPGGALSLVCKASGFDFSSYQMNWIRQAPGKGLEWVAA
INKFGTSTSRGAAVKGRVTISRDDGQSTVRLQLNNLRSEDTATYFCAKSA
YGSCASGSWCSAASIDAWGHGTEVIVSSTSGQAGQHHHHHHGAYPYDVPD
YAS
scFv
(SEQ ID NO: 33)
KWYGWYQQKAPGSAPVTLIYDNTNRPSDIPSRFSGSASGSTATLTITGVQ
VEDEAVYFCGGYDGSTDAGIFGAGTTLTVLGQSSRSSAVTLDESGGGLQT
PGGALSLVCKASGFDFSSYQMNWIRQAPGKGLEWVAAINKFGTSTSRGAA
VKGRVTISRDDGQSTVRLQLNNLRSEDTATYFCAKSAYGSCASGSWCSAA
SIDAWGHGTEVIVSSTSGQAGQHHHHHHGAYPYDVPDYAS

### 2.9. Immobilised Metal Affinity Chromatography (IMAC) Purification

For further analysis by HPLC and Surface Plasmon Resonance (SPR), the AE8 clone was purified by immobilised metal affinity chromatography (IMAC). A single colony of the AE8 clone was sub-cultured into 5 mls of 2×TY containing 100 μg/ml carbenicillin and 1% (w/v) glucose and grown overnight at 37° C. Five hundred uls of the overnight culture was inoculated into 500 mls of terrific-broth (TB; tryptone 13.3 g/l, yeast extract 26.6 g/l and glycerol 0.44%, (v/v)) that contained 1×505 medium (0.5% (v/v) glycerol, 0.05% (v/v) glucose final concentration), 50 mls potassium phosphate solution (KH₂PO₄ 2.31 g/l, K₂HPO₄ 12.54 g/l), 1 mM MgSO₄ and 100 μg/ml carbenicillin. The culture was incubated at 37° C. at 240 rpm until an approximate OD₆₀₀ of 0.6 was reached. The culture was then induced with 1 mM IPTG and incubated at 30° C. overnight at 240 rpm. The following day, the culture was centrifuged at 4000 rpm for 10 minutes at 4° C. and the pellet was completely re-suspended in 30 mls of ice-cold sonication buffer (1×PBS, 0.5M NaCl and 20 mM imidazole) and then aliquoted (1 ml) into 1.5 ml Eppendorf tubes. Each individual sample was sonicated on ice for 45 seconds (40% amplitude) with 6 sec pulses for 3 minutes. The samples were then centrifuged at 14,000 rpm for 10 minutes at 4° C. The lysates were pooled, filtered through a 0.2 μM filter and stored at 4° C. All IMAC purifications were performed using PD-10 columns (GE Healthcare, U.K.). Two millilitres of Ni-NTA resin (Qiagen, USA) were added to the column and allowed to form a packed bed. After equilibration with 30 mls of running buffer (sonication buffer containing 1% (v/v) Tween), the pooled lysate was then added to the column and the flow-through was collected and stored at 4° C. The column was subsequently washed with 30 mls of running buffer and the bound scFv was eluted by adding 20 mls of 100 mM sodium acetate (pH 4.4). 400 μl volumes of eluent were added to 50 μl of 10×PBS (pH 7.2) and 50 μl of 100 mM NaOH before mixing. Individual fractions were tested for the presence of protein by quantification at 280 nm with the Nanodrop ND1000 spectrophotometer. Those fractions that contained the eluted scFv were pooled and concentrated using a 5000 Da molecular weight cut-off (MWCO) buffer exchange column (Sartorius, Germany). The scFv-containing sample was concentrated to a volume of 500 μl by centrifugation (4000 rpm) at 4° C. Five mls of 1×PBS were subsequently added to the column and, after an overnight incubation at 4° C., the sample was buffer exchanged and re-concentrated by centrifugation until the final volume was approximately 200 μl. Protein concentration was determined by quantification at 280 nm using a Nanodrop ND1000 spectrophotometer (Labtech International, U.K.).

2.10. SEC-HPLC Analysis of scFv Clone AE8

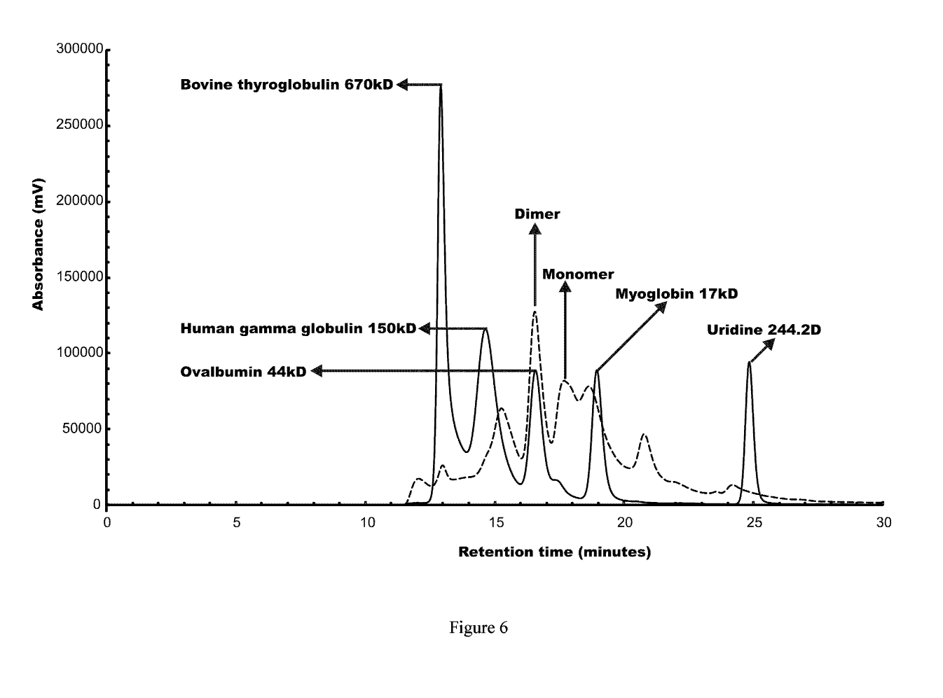

HPLC size exclusion chromatography (SEC-HPLC) was used to determine the species composition, apparent molecular weight and to purify the monomeric fraction of the recombinant AE8 scFv. A Shimadzu LC system (Shimadzu Corporation, Japan), equipped with a Shimadzu CBM-20A controller, Shimadzu LC-20AB pumps, Shimadzu SPD-20A UV-Vis spectrophotometric detector, Shimadzu SIL-20A autosampler, Shimadzu FRC-10A fraction collector, Shimadzu CTO-20AC column oven and Shimadzu's LCsolution software for data handling. The experiments were carried out using the size exclusion Bio-Sep-SEC-52000 column (Phenomenex; 300×7.8 mm) protected with a guard column (Phenomenex; 35×7.8 mm). The HPLC system was operated isocratically at room temperature using filtered and degassed 1×PBS (pH 7.2) as the mobile phase. Prior to sample analysis, the column was equilibrated for 45 minutes by gradually increasing the flow rate in increments of 0.1 ml/minutes. All samples (20 μl) were diluted in 1×PBS (pH 7.2) and assayed at a flow rate of 0.5 mls/minutes with UV detection (280 nm). The following protein standards (Agilent, USA) were used: bovine thyroglobulin (670 kD), Human gamma globulin (IgG; 150 kD), ovalbumin (44 kD) and myoglobin (17 kD). Samples were interspersed with water blanks to ensure that all residual protein was eluted. The monomeric AE8 scFv was isolated with the Bio-Sep-SEC-52000 HPLC column by the collection of several fractions between 17.4 and 18.2 minutes at a flow rate of 0.5 ml/minute (FIG. 6).

2.11. FPLC Analysis of scFv Clone AE8

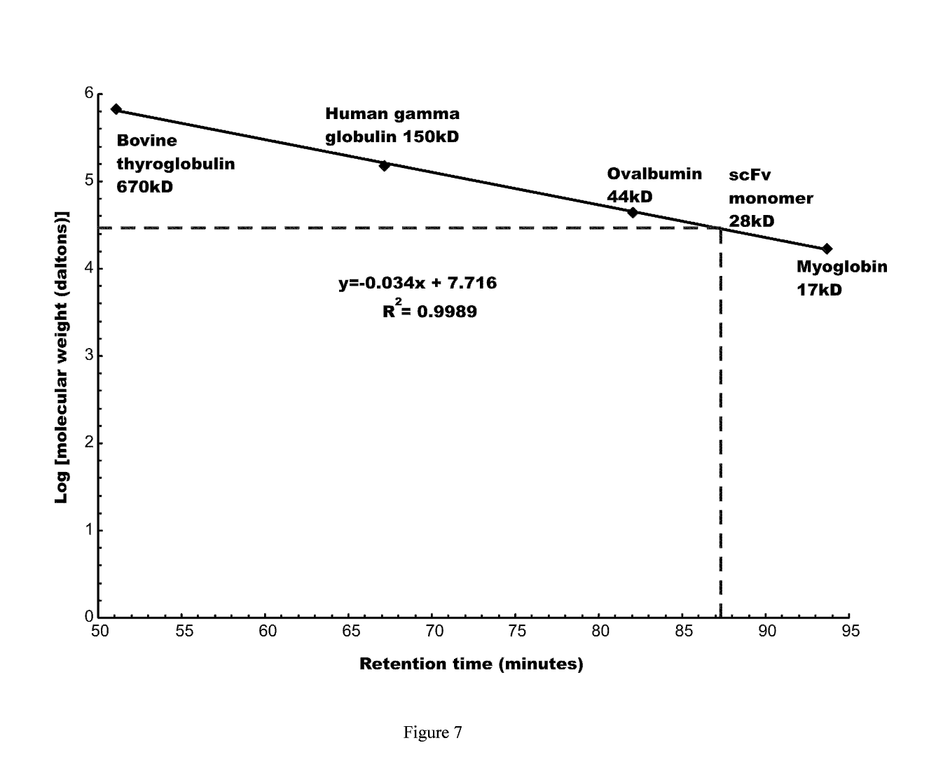

Fast protein liquid chromatography (FPLC) was used to estimate the molecular weight of the AE8 protein. The ÄKTA™ Explorer 100 system (GE Healthcare, USA) equipped with a UV-900 monitor, monitor pH/C-900, sample pump, fraction collector frac-950 and UNICORN™ software for data handling was used for protein analysis. 100 μl of the AE8 sample was applied to a HiLoad™ 16/60 Superdex™ 200 Prep-grade FPLC column using filtered and degassed 1×PBS (pH 7.2) at a flow rate of 1 ml/min. The following protein standards (Agilent, USA): bovine thyroglobulin (670 kD), Human gamma globulin (IgG; 150 kD), ovalbumin (44 kD) and myoglobin (17 kD) were used for the molecular weight estimation of the scFv (FIG. 7).

### 3.0 Surface Plasmon Resonance Analysis of the AE8 Clone Using the BIAcore® 3000 Biosensor

Analysis of the binding and kinetic properties of the AE8 clone was performed using the BIAcore® 3000 biosensor which monitors ‘label-free’ biomolecular interactions in ‘real-time’ using the phenomenon of surface plasmon resonance (SPR). The basic assay format for AE8 SPR analysis was as follows: neutravidin was immobilised on the dextran surface of a BIAcore® CM5 chip, biotinylated polyacrylamide Neu5Gc conjugate was then passed over and captured by the neutravidin, after which the scFv was passed over the surface to check for binding to the sugar. As a negative control, the scFv was also passed over a neutravidin surface which had no biotinylated sugar.

### 3.1 Pre-Concentration, Immobilisation of Neutravidin on a Carboxy-Methylated Dextran Chip and Capture of Biotinylated-Neu5Gc Polyacrylamide (PAA).

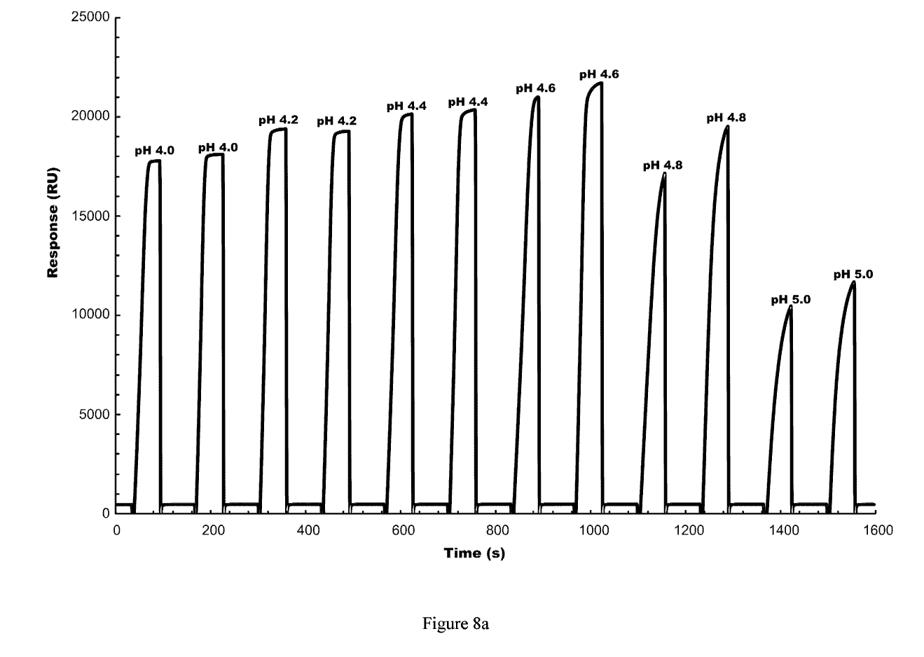

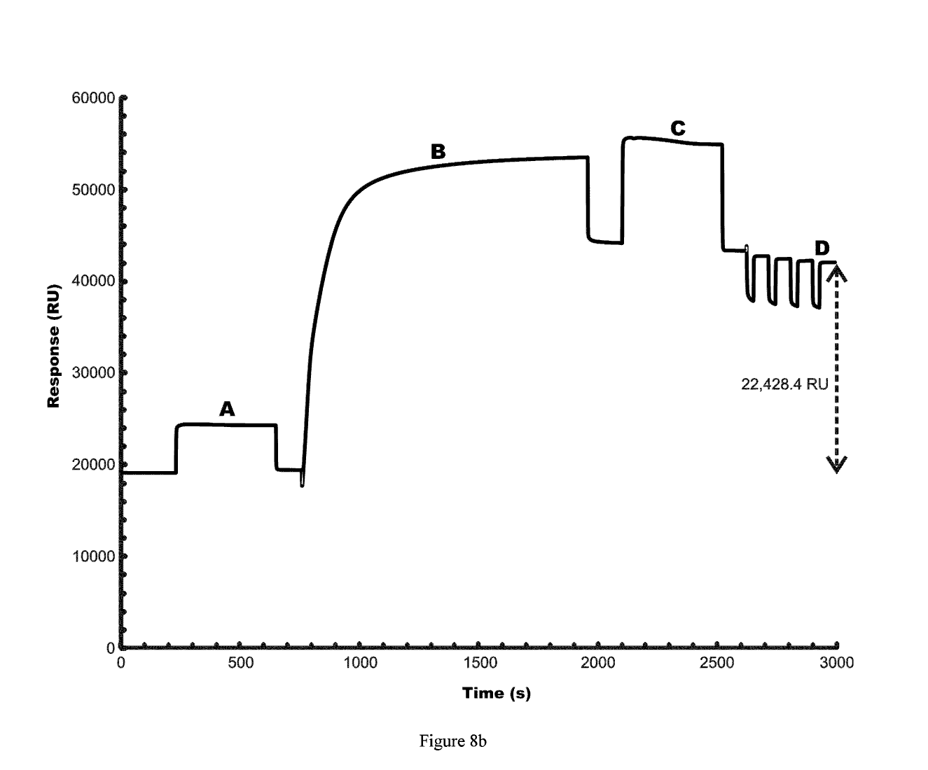

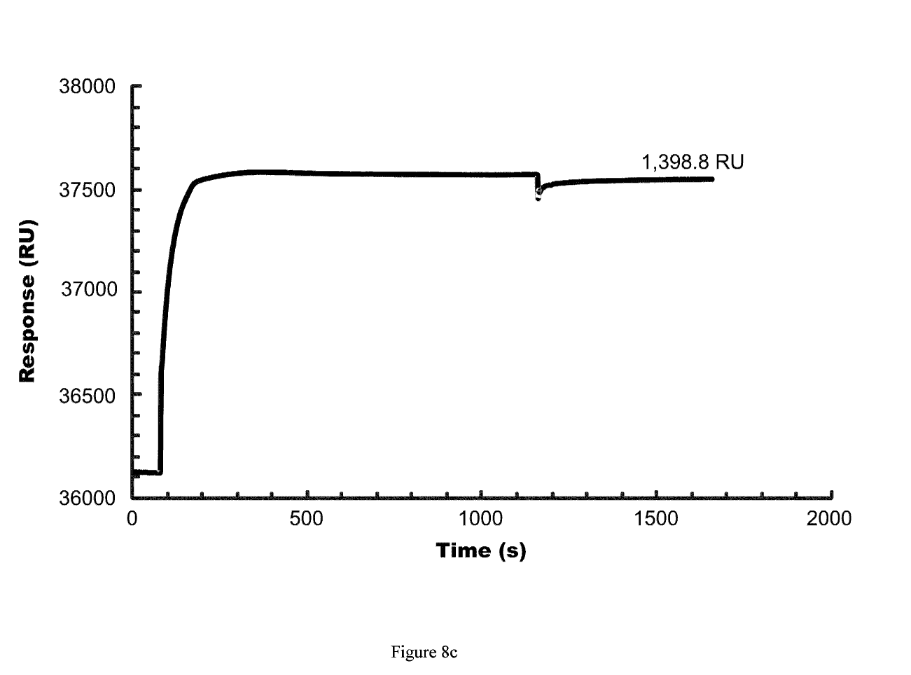

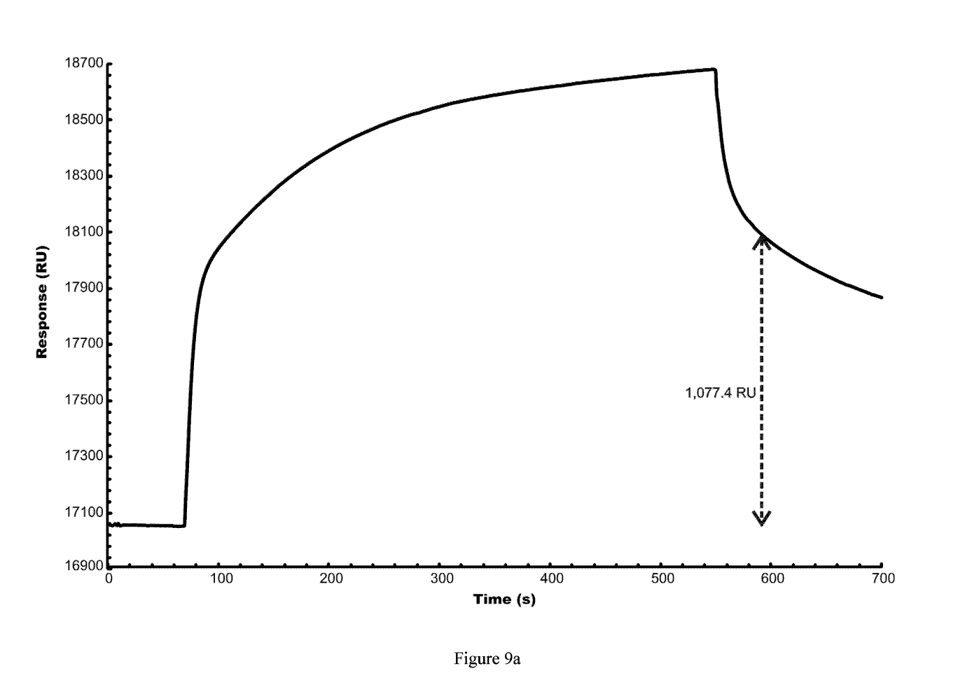

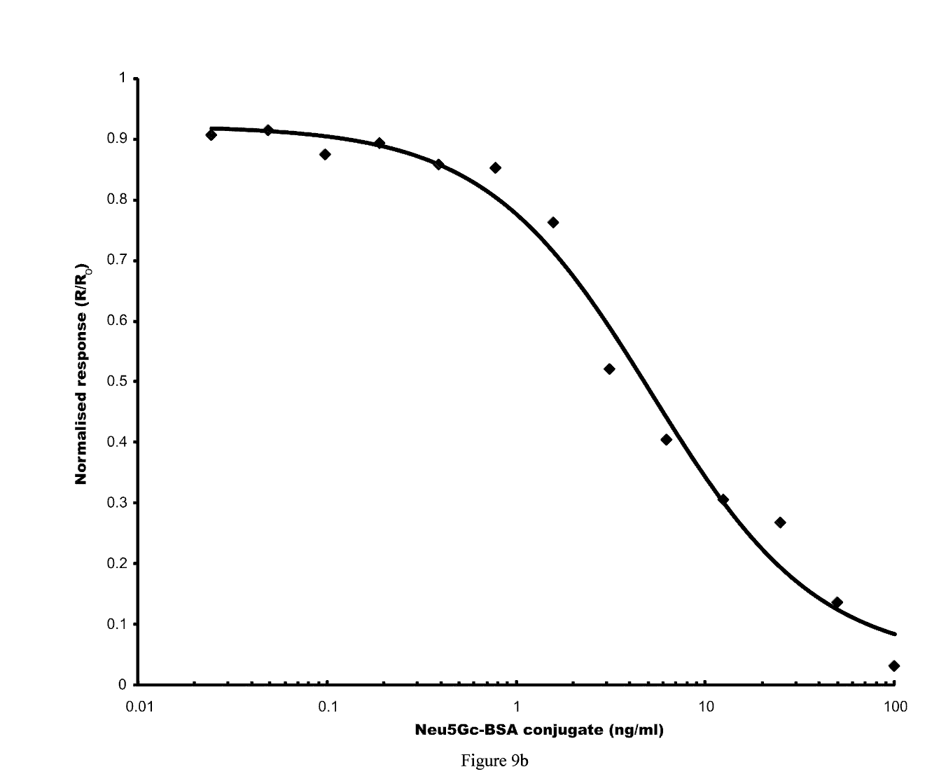

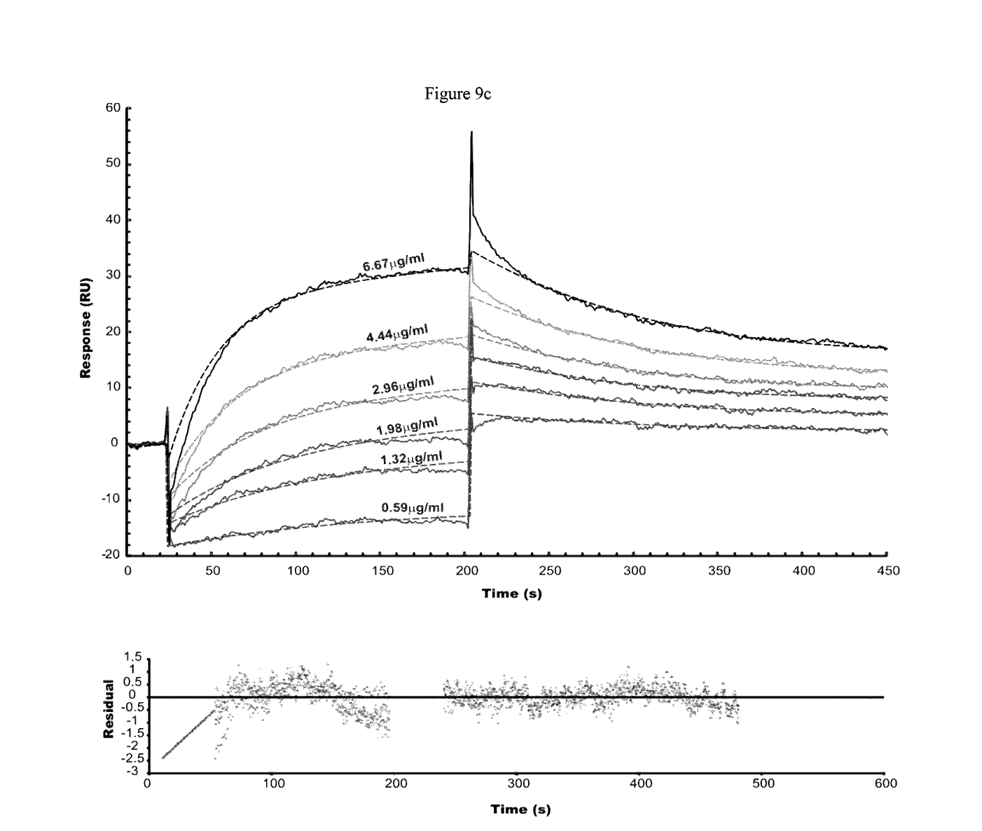

For all BIAcore® 3000 (GE Healthcare, Sweden) experiments, the running buffer used was filtered and degassed HEPES buffered saline pH 7.4 (HBS: 50 mM NaCl, 10 mM HEPES, 3.4 mM EDTA and 0.05% (v/v) Tween-20). 50 μg/ml solutions of neutravidin (Thermo Fisher Scientific, USA) were prepared in 10 mM sodium acetate (Sigma-Aldrich, USA) buffers that had been adjusted with 10% (v/v) acetic acid (Sigma-Aldrich, USA) to pH values 4.0, 4.2, 4.4, 4.6, 4.8, and 5.0. 20 μl of protein at each respective pH was sequentially passed over the underivatised carboxy-methylated dextran sensor chip surface (CM5, GE Healthcare, Sweden) at a flow-rate of 10 μl/minute. A pH of 4.6 was determined to be the optimal pH for neutravidin immobilisation as this yielded the largest change in response units (RU). Neutravidin was immobilised on the CM5 chip with the following protocol: 70 μl of 400 mM of 1-ethyl-3-[3-dimethylaminopropyl]carbodiimide hydrochloride (EDC) (GE Healthcare, Sweden) was mixed with 70 μl of 100 mM N-hydroxysuccinimide (NHS) (GE Healthcare, Sweden) and injected over the sensor chip surface for 10 minutes at a flowrate of 10 μl/minute. A 50 μg/ml solution of neutravidin was prepared in 10 mM sodium acetate (Sigma-Aldrich, USA), pH 4.6 and injected over the activated chip surface for 24 minutes at a flow-rate of 10 μl/minute. Unreacted NHS ester groups were capped and loose, non-covalently attached proteins were removed by injection of 1M ethanolamine hydrochloride (GE Healthcare, Sweden), pH 8.5, for 11 minutes. Four 30 second sequential pulses of 5 mM NaOH at a flow-rate of 10 μl/minute were used to remove any other loosely bound material. After neutravidin immobilisation (FIG. 8b), a 100 μg/ml solution of biotinylated-Neu5Gc-PAA in HBS was passed over the chip surface at 10 μl/min for 20 minutes (FIG. 8c).

### 3.2. SPR Analysis of the AE8 Clone.

The sialic acid binding ability of the AE8 clone was assessed with the previously prepared Neu5Gc sensor chip. A 1 in 100 dilution of the IMAC purified AE8 clone in FIBS was simultaneously passed over flow cells 1 and 2 of the sensor chip at a flow rate of 10 μl/min for 7 minutes. Following on-line reference subtraction (2-1), the sensorgram indicated a response increase of 1,077.4 RU above baseline. Bound antibody was dissociated with a 30 second pulse of 10 mM NaOH and the baseline was restored with the injection of HBS running buffer over the chip surface. The experiment was repeated five times and, on each occasion, the AE8 Neu5Gc binding response was greater than 1000 RU.

### 3.3 Solution-Phase Neu5Gc-Binding Assay

To assess the ability of the anti-sialic acid scFv to bind the sialic acid conjugate in solution-phase, an inhibition binding assay was performed. The purified AE8 scFv was diluted 1 in 2000 in HBS buffer (pH 7.4). The Neu5Gc-BSA conjugate was also diluted in HBS buffer (pH 7.4) to the following concentrations: 2000 ng/ml, 1000 ng/ml, 500 ng/ml, 250 ng/ml, 125 ng/ml and 62.5 ng/ml. 100 μl of the AE8 sample was mixed with 100 μl of each of the Neu5Gc-BSA conjugate dilutions to yield the following free conjugate working concentrations: 1000 ng/ml, 500 ng/ml, 250 ng/ml, 125 ng/ml, 62.5 ng/ml and 31.25 ng/ml. The zero conjugate sample contained 100 μl of 1 in 2000 dilution of the purified AE8 scFv in HBS buffer (pH 7.4) and 100 μl of HBS buffer (pH 7.4). Samples were incubated for 1 hour at 37° C. and then injected (40 μl), in random order, over flow cells 1 and 2 of the Neu5Gc chip at a flow rate of 10 μl/minute for 4 minutes and the change in response recorded. Bound antibody was removed by injection of 5 μl of 5 mM NaOH at a flow rate of 10 μl/minute for 30 seconds. The amount of free antigen necessary to cause 50% displacement of antibody (IC₅₀) was 5.7 ng/ml.

### 3.4 Preliminary SPR Kinetic Studies on the AE8 Clone

SPR was used to determine the association and dissociation rate constants of the anti-sialic acid scFv. The rate constants were fitted with a pre-defined fitting algorithm using the Biaevaluation 4.1 software. To avoid mass-transfer limited binding, a smaller quantity (1 μg/ml) of neutravidin (<10,000 RU) was immobilised (see section 3.1) on the sensor chip surface. Subsequently, for the capture step, a 40 ng/ml solution of biotinylated-Neu5Gc-PAA in EMS buffer (pH 7.4) was passed over the chip surface at 10 μl/min for 1 minute. A final level of 28.6 RU of captured biotinylated-Neu5Gc-PAA was achieved. Furthermore, to rule out the contribution of avidity in the determination of the rate constants, only the monomeric HPLC-purified fraction of AE8 was used for Biacore kinetic analysis. The rate constants were calculated using different concentrations (6.67 μg/ml, 4.44 μg/ml, 2.96 μg/ml, 1.98 μg/ml, 1.32 μg/ml, 0.88 μg/ml, 0.59 μg/ml and 0 μg/ml) of monomeric scFv diluted in HBS buffer (pH 7.4). The kinject command was used to inject 90 μl of each sample over flow cells 1 and 2 of the Neu5Gc sensor chip, at a flow rate of 30 μl/minute for 3 minutes with a dissociation time of 12 minutes. The zero scFv sample was analysed twice and all samples were run in random order. To reflect the PBS composition of the HPLC eluted monomeric scFv, the zero scFv sample contained 1×PBS (pH 7.2) diluted 1 in 10 in HBS buffer (pH 7.4). Bound antibody was removed by injection of 5 μl of 1.25 mM NaOH at a flow rate of 30 μl/minute for 10 seconds. All sensorgrams were reference-subtracted from flow cell 1, which contained a blank dextran surface. In addition, to remove systematic anomalies a blank run consisting of a zero concentration of the scFv samples was subtracted from each of the sensorgrams.

### REFERENCES

1. Kelm, S., and Schauer, R. (1997). Sialic acids in molecular and cellular interactions. Int. Rev. Cytol. 175:37-240.
2. Schauer, R. (2000). Achievements and challenges of sialic acid research. Glycoconjugate J. 17:495-499.
3. Varki, A. (2008). Sialic acids in human health and disease. Trends Mol. Med. 14:(8)351-60.
4. Varki, A., Freeze, H., H., Manzi, A., E. (2001). Overview of glycoconjugate analysis. Curr. Protoc. Protein Sci. 12:12.1.
5. Varki, N., M., and Varki, A. (2007). Diversity in cell surface sialic acid presentations: implications for biology and disease. Lab Invest. 87(9):851-7.
6. Angata, T. and Varki, A. (2002). Chemical diversity in the sialic acids and related α-keto acids: an evolutionary perspective. Chem. Rev. 102:439-469.
7. Slovin, F., S., Keding, J., S., Ragupathi., G. (2005). Carbohydrate vaccines as immunotherapy for cancer. Immunol. Cell Biol. 83:418-428.
8. Sakai, K., Shimizu, Y., Chiba, T., Matsumoto-Takasaki, A., Kusada, Y., Zhang, W., Nakata, M., Kojima, N., Toma, K., Takayanagi, A., Shimizu, N., Yoko Fujita-Yamaguchi, Y. (2007). Isolation and Characterization of Phage-Displayed Single Chain Antibodies Recognizing Nonreducing Terminal Mannose Residues. 1. A New Strategy for Generation of Anti-Carbohydrate Antibodies. Biochemistry 46(1):253-262.
9. Deng, S., Roger M., C., Sadowska, J., Michniewicz, J., Martin, Y., N., Bundle, R., D., Narang, A., S. (1994). Selection of Antibody Single-chain Variable Fragments with Improved Carbohydrate Binding by Phage Display. J. Biological Chemistry 269(13):9533-38.
10. Smith, G., P. (1985). Filamentous fusion phage: novel expression vectors that display cloned antigens on the virion surface. Science 228(4705):1315-7.
11. Vaughan, T., J., Osbourn, J., K., Tempest, P., R. (1998). Human antibodies by design. Nature Biotechnology 16(6):535-9.
12. Schoonbroodt, S., Steukers, M., Viswanathan, M., Frans, N., Timmermans, M., Wehnert, A., Nguyen, M., Ladner, R., C., Hoet, R., M. (2008). Engineering antibody heavy chain CDR3 to create a phage display Fab library rich in antibodies that bind charged carbohydrates. J. Immunol. 181(9):6213-21.

What is claimed is:

1. A method of generating and isolating a recombinant high affinity anti-sialic acid antibody molecule, the method comprising: immunising a host with an immunogen comprising a conjugate of a sialic acid and a carrier protein to generate an anti-sialic acid polyclonal serum, wherein the host and conjugate are chosen such that the host glycome is deficient in the sialic acid; isolating a sample of RNA from the immunised avian host; generating and screening a library of recombinant antibody molecules from the RNA sample; and isolating a recombinant high affinity anti-sialic acid antibody molecule, wherein the conjugate comprises at least 2 sialic acid molecules bound to one carrier protein.

2. A method as claimed in claim 1 in which the conjugate comprises at least 10 sialic acid molecules bound to one carrier protein.

3. A method as claimed in claim 1 in which the sialic acid is either Neu5Gc or Neu5Ac.

4. A method as claimed in claim 1 in which the carrier protein is a serum albumin protein.

5. A method as claimed in claim 1 in which the sialic acid is Neu5Gc or Neu5Ac, and the conjugate comprises at least five molecules of Neu5Gc or Neu5Ac bound to one molecule of carrier protein.

6. A method as claimed in claim 1 in which the sialic acid is conjugated to the carrier protein by a linker comprising a hydrocarbon chain having at least five carbon atoms.

7. A method as claimed in claim 1 in which the antibody molecule is selected from the group consisting of whole antibodies, scFv fragments, and Fab fragments.

8. A method as claimed in claim 1 in which the sialic acid is Neu5Gc and the host is avian.

9. A method as claimed in claim 8 in which the avian host is a member of the Gallus family.

10. An anti-sialic acid polyclonal serum obtainable by immunising a host with a conjugate of sialic acid and carrier protein, wherein the host glycome is deficient in the sialic acid.

11. An anti-sialic acid polyclonal serum as claimed in claim 10 in which the conjugate comprises at least two sialic acid molecules conjugates to one carrier protein molecule.

12. A conjugate of sialic acid and a carrier protein in which the conjugate comprises at least two sialic acid molecules and one carrier protein molecule.

13. A conjugate as claimed in claim 12 having at least ten sialic acid molecules conjugated to one carrier protein molecule.

14. A conjugate as claimed in claim 12 in which the sialic acid is Neu5Gc or Neu5Ac6

15. An isolated, recombinant anti-sialic acid antibody molecule or fragment having a nanomolar binding affinity to sialic acid.

16. An isolated, recombinant anti-sialic acid antibody molecule according claim 15, the antibody molecule comprising a light chain variable region having a CDRL1 region according to SEQ ID NO: 12, 13 or 14, a CDRL2 region according to SEQ ID NO: 7 or 8, and a CDRL3 region according to SEQ ID NO'S: 9, 10 or 11, and a heavy chain variable region having a CDRH1 region according to SEQ ID NO: 15, 16 or 17, a CDRH2 region according to SEQ ID NO: 18 or 19, and a CDRH3 region according to SEQ ID NO'S: 20 or 21, or a functional variant of the antibody molecule.

17. An isolated, recombinant anti-sialic acid antibody molecule according to claim 16 in which the light chain variable region comprises a CDRL1 region according to SEQ ID NO: 14, a CDRL2 region according to SEQ ID NO: 8, a CDRL3 region according to SEQ ID NO: 11, and the heavy chain variable region comprises a CDRH1 region according to SEQ ID NO: 17, a CDRH2 region according to SEQ ID NO: 19, and a CDRH3 region according to SEQ ID NO: 21, or a functional variant of the antibody molecule.

18. An isolated, recombinant anti-sialic acid antibody molecule according to claim 15 in which the light chain variable region comprises a sequence according to SEQ ID NO: 22, or a functional variant thereof, and the heavy chain variable region comprising a sequence according to SEQ ID NO: 23, or a functional variant thereof.

19. An isolated, recombinant anti-sialic acid antibody molecule according to claim 15, in which the light chain variable region comprises a sequence according to SEQ ID NO: 24, or a functional variant thereof, and the heavy chain variable region comprising a sequence according to SEQ ID NO: 25, or a functional variant thereof.

20. An isolated, recombinant anti-sialic acid antibody molecule according to claim 15, in which the light chain variable region comprises a sequence according to SEQ ID NO: 26, or a functional variant thereof, and the heavy chain variable region comprises a sequence according to SEQ ID NO: 27, or a functional variant thereof.

21. An isolated, recombinant anti-sialic acid antibody molecule according to claim 15, in which the light chain variable region comprises a sequence according to SEQ ID NO: 28, or a functional variant thereof, and the heavy chain variable region comprises a sequence according to SEQ ID NO: 29, or a functional variant thereof.

22. An isolated, recombinant anti-sialic acid antibody molecule according to claim 15 comprising a sequence selected from the group consisting of: SEQ ID NO: 30; 31; 32; and 33.
